(19) United States
(12) Patent Application Publication
(10) Pub. No.: US 2026/0363674 A1
(43) Pub. Date: Nov. 19, 2026

(54) METHOD FOR ESTIMATING A CONCENTRATION OF GAS EMITTED BY A MEDIUM

(71) Applicants: COMMISSARIAT A L'ENERGIE ATOMIQUE ET AUX ENERGIES ALTERNATIVES, Paris (FR); INSTITUT POLYTECHNIQUE DE GRENOBLE, Grenoble (FR); UNIVERSITE GRENOBLE ALPES, Saint Martin D'Heres (FR); CENTRE NATIONAL DE LA RECHERCHE SCIENTIFIQUE, Paris 16 (FR)

(72) Inventors: Pierre GRANGEAT, Grenoble Cedex 09 (FR); Maria-Paula COMSA, Grenoble Cedex 09 (FR); Anne KOENIG, Grenoble Cedex (FR); Ronald PHLYPO, Saint Martin D'Heres (FR)

(73) Assignees: COMMISSARIAT A L'ENERGIE ATOMIQUE ET AUX ENERGIES ALTERNATIVES, Paris (FR); INSTITUT POLYTECHNIQUE DE GRENOBLE, Grenoble (FR); UNIVERSITE GRENOBLE ALPES, Saint Martin D'Heres (FR); CENTRE NATIONAL DE LA RECHERCHE SCIENTIFIQUE, Paris 16 (FR)

(21) Appl. No.: 19/108,002

(22) PCT Filed: Aug. 27, 2023
(86) PCT No.: PCT/EP2023/073458

§ 371 Date: Feb. 28, 2025

(30) Foreign Application Priority Data
Aug. 28, 2022 (FR) FR2208595
Dec. 3, 2022 (FR) FR2212730

(51) Int. Cl. A61B 10/00; A61B 5/00

(52) U.S. Cl. CPC A61B 10/00; A61B 5/725; A61B 2010/0083

(57) ABSTRACT

A method of estimating a content of a gas of interest in a medium, by means of a measuring device, intended to be disposed against the medium, the device extending between a contact face, intended to be applied against the medium and a distal end, the device comprising a side wall, extending between the contact face and the distal end. The device includes at the contact face, at least one inlet opening, configured to collect the gas of interest emitted by the medium, the inlet opening being made through the contact face; a measurement chamber, comprising a gas sensor, the gas sensor being configured to measure a concentration of gas of interest flowing through the measurement chamber; and a collection chamber connected to the measurement chamber and delimited by the side wall, the collection chamber having at least one lateral opening through the side face for admitting ambient air into the collection chamber

### TECHNICAL FIELD

The technical scope of the invention is the measurement of a gas released by a medium using a compact device. The compact device is applied against the medium. The medium may be the skin of a living being. The process enables the content of a gas released by the medium to be estimated. The gas may be carbon dioxide, for example, for capnometry applications. This involves estimating the content of carbon dioxide dissolved in the blood. The medium can be a liquid medium, such as water or liquid manure. The gas may be carbon dioxide or methane. The medium can also be a plant medium, in which case the device can be used to study the respiration of the medium

### PRIOR ART

Some respiratory diseases affect gas exchange between blood and exhaled air. Blood contains dissolved gases including oxygen and carbon dioxide (CO₂), whose respective partial pressures reflect the gas exchanges taking place in the lungs and organs.

To assess the concentration dissolved CO₂ in the blood, a blood sample may be taken. This is an invasive method, which can be painful and difficult to apply, particularly in neonatology. Moreover, it can only be applied on an ad hoc basis. Despite these drawbacks, its reliability has been validated by the medical profession, and it is a reference method. Another method consists in estimating the CO₂ content of blood non-invasively, by measuring the partial pressure of CO₂ diffusing through tissues, particularly the skin. This method is known as transcutaneous capnometry.

In the blood, carbon dioxide is dissolved as a molecular species (CO₂), and as two ionic species: carbonate ions CO₃²⁻ and bicarbonate ions HCO₃⁻. The balance between these species depends on the pH of the blood, as carbonate and bicarbonate ions are in equilibrium with hydrogen ions H⁺. Thus, the concentration of carbonate or bicarbonate ions influences blood pH. An increase in the concentration of dissolved CO₂ (hypercapnia) results in an increase in the quantity of carbonate and bicarbonate ions, and, by equilibrium, hydrogen ions, leading to a decrease in blood pH, or acidosis. An increase in CO₂ concentration can occur when the elimination of CO₂ by the respiratory tract is insufficient, for example in the case of chronic obstructive pulmonary disease (COPD) or infectious diseases affecting the lungs, one example being an infection due to COVID 19.

Conversely, a decrease in the concentration of dissolved CO₂ (hypocapnia) lowers the concentration of hydrogen ions, resulting in an increase in pH, or alkalosis. Hypocapnia can be caused, for example, by hyperventilation associated with an increase in respiratory rate. The occurrence of acidosis or alkalosis may have consequences for metabolism. Thus, blood CO₂ concentration is an important vital parameter, which should be monitored regularly for certain patients at risk.

CO₂ concentration monitoring can also be applied to patients in intensive care, or to newborns placed in incubators. It can also be used to monitor exercise, as physical activity promotes the production of carbon dioxide.

This type of analysis, based on transcutaneous measurements, was introduced in the 1980s. Transcutaneous measurements enable continuous monitoring, for example, to track the immediate effects of therapeutic treatment on blood CO₂ concentrations. It can also be used to determine when more precise quantification by blood sampling is preferable. This shows that invasive and non-invasive methods can be complementary: one is precise and punctual, while the other can be implemented on a continuous basis for telemonitoring.

WO2017/023500 describes a device for measuring a gas, in this case NO, emitted through the skin. The device comprises a collection chamber with a lateral opening. The device has a measurement chamber arranged downstream of the collection chamber. As a result, the gas flowing through the measurement chamber contains a significant proportion of external air, which adversely affects sensitivity.

JP2010148692 describes a device for measuring a gas, in this case H₂, emitted through the skin. The device has a lateral opening allowing either the admission of a gas into a collection chamber, or the evacuation of the gas. A compact device for measuring transcutaneous CO₂ has already been described in WO2020/249466. This is a non-invasive measuring device, worn by a user, for estimating a concentration of a gas of interest emitted transcutaneously, the gas of interest being carbon dioxide, for example. The circulation of the gas in the device enables collection of the gas of interest. The gas of interest propagates through the device by convection, under the effect of a heat source. The device has a contact face, intended to be applied against the user. The device also includes a measurement chamber, comprising a sensor for the gas of interest, typically CO₂. In order for the temperature rise to result in convection, it is preferable for the measurement chamber to be positioned above the contact face

The inventors propose a refinement of the device described in WO2020/249466, aimed at improving certain performances, in particular temporal response performances. The inventors also propose a method for estimating, with the aid of the device, the concentration of an analyte in the medium in front of which the device is arranged.

### SUMMARY OF THE INVENTION

An object of the invention is a method for estimating a content of a gas of interest in a medium, by means of a measuring device, intended to be arranged against the medium, the measuring device extending between a contact face, intended to be applied against the medium and a distal end, the measuring device comprising a side wall, extending between the contact face and the distal end, the measuring device comprising:
at the contact face, at least one inlet opening, configured to collect the gas of interest emitted by the medium, the inlet opening being formed through the contact face;
a measurement chamber, comprising a gas sensor, the gas sensor being configured to measure a concentration of gas of interest flowing through the measurement chamber;
a collection chamber connected to the measurement chamber and delimited by an opening in the side wall, the collection chamber having at least one lateral opening through the side face or top wall of the collection chamber for admitting ambient air into the collection chamber;
the device being such that:
the measurement chamber is located between the contact face and the collection chamber;
the device comprises a drive means, configured to drive air from the collection chamber towards a discharge opening, the air drive inducing a transport of the gas of interest from the contact face to the collection chamber, through the measurement chamber:
the method comprising:
a) measuring the concentration of the gas of interest in the measurement chamber;
b) by means of a processing unit, modeling of the transport of the gas of interest between the medium and the collection chamber, the modeling taking into account the diffusion of the gas from the medium to the collection chamber through the contact face and the device, as well as the convection of the gas in the collection cell resulting from the entrainment produced by the drive means;
c) from the measurement resulting from step a), and taking into account the model resulting from step b), estimating the content of the gas of interest in the medium.

In one embodiment,
the device is spatially discretized according to a spatial mesh, defining mesh points between the contact face and the collection chamber;
the model is a discretized spatio-temporal model, so as to estimate a content of the gas of interest at different points in the mesh, and at different times.

The method may be such that step b) comprises:
modeling the diffusion of the gas of interest through the measurement chamber;
modeling the diffusion and convection of the gas of interest in the collection chamber.

Step b) involves modeling the transport of the gas of interest in the medium.

Preferably, step c) implements a recursive estimator. This may be a linear recursive estimator. The linear recursive estimator can be a Kalman filter.

According to one application, the medium is the skin of a user, the skin extending over a blood vessel. Step b) may then comprise:
(i) from the gas concentration of interest in the measurement chamber resulting from step a), estimating a transcutaneous gas concentration of interest;
(ii) from the transcutaneous gas concentration of interest resulting from sub-step (i), estimating a concentration or partial pressure of gas of interest dissolved in the user's blood.

The process can comprise modeling the transport of the gas of interest emitted by the medium through the device.

The medium to be analyzed may be solid or liquid.

The drive means may be an air propeller or an air aspirator.

Modeling can be based on a two-dimensional model, with the medium, measurement chamber and collection chamber discretized according to different mesh points distributed along an axis parallel to the contact face and an axis perpendicular to the contact face.

The invention will be better understood on reading the examples of embodiments presented, in the following description, in connection with the figures listed below.

### FIGURES

FIG. 1A shows a first embodiment of a device according to the invention.

FIG. 1B shows the flow inside the device.

FIG. 2A shows the main components of a gas sensor, arranged in the device's measurement chamber. The view is taken in a plane perpendicular to a transverse axis.

FIG. 2B shows the main components of a gas sensor, based on infra-red light absorption, arranged in the device's measurement chamber. The view is taken in a plane parallel to the transverse axis.

FIG. 3 shows a breakdown of the measuring device and the medium to be analyzed into compartments.

FIG. 4 shows a discretization of each compartment shown in FIG. 3.

FIG. 5 illustrates the main steps in implementing a process according to the invention.

FIGS. 6A, 6B and 6C show an estimate of a CO2 concentration at different sampling points when the concentration in the blood follows a square wave variation.

FIG. 6D shows an estimate of the CO2 concentration measured by the device's gas sensor, and an application of white noise to said concentration. The application of white noise

FIGS. 7A, 7B, 7C, 7D show an estimate of a CO2 concentration at different sampling points when the concentration measured by the sensor follows the noisy concentration simulated in FIG. 6D.

FIG. 7E shows a comparison between the square-wave blood concentration, which represents the actual value, and the blood concentration estimated by the recursive estimator.

FIG. 8 shows a two-dimensional discretization of each compartment.

FIG. 9 illustrates an interface separating two adjacent compartments.

### DESCRIPTION OF PARTICULAR EMBODIMENTS

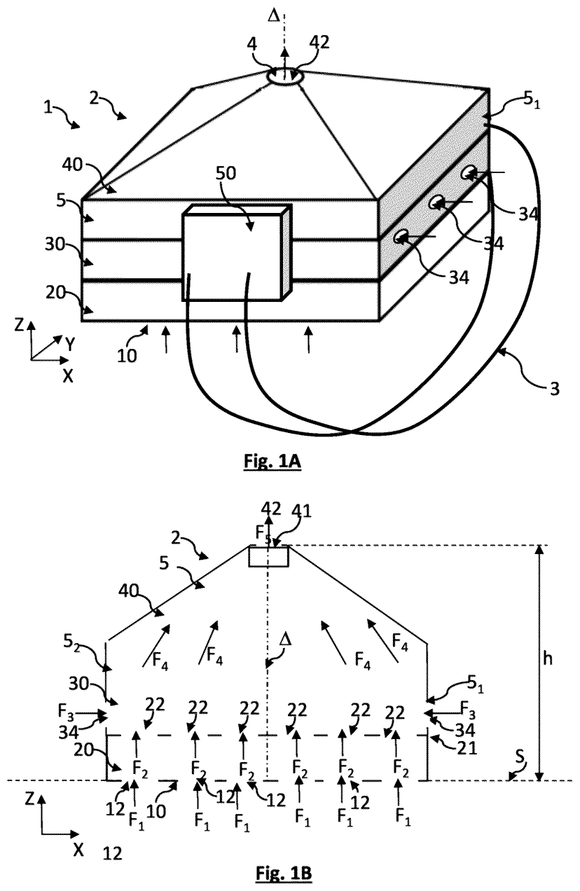

FIGS. 1A and 1B are general views of an example of a device 1 according to the invention. The device 1 is intended to be placed in contact with a medium to be analyzed. In this example, the medium is the skin S of a human or animal user. The device comprises a main body 2 and a fastening element 3, the latter being, in this example, a bracelet. Alternatively, the main body can be placed in contact with an earlobe or a finger. The holder can be a clip or integrated into a headset. More generally, the support is configured to hold the main body in contact with the medium to be analyzed.

The device is intended to estimate a concentration of a gas of interest emanating from a user's skin. By gas of interest is meant a gas whose concentration in a living human or animal body, and more particularly in the blood, is to be determined. In the non-limiting example described below, the gas of interest is carbon dioxide, whose content in the user's blood is to be estimated. Other possibilities include, but are not limited to, oxygen, ethyl alcohol, carbon monoxide, methane, nitric oxide, acetone or isoprene, hydrogen, certain drugs or volatile substances.

The main body 2 comprises a contact face 10, intended to be affixed to the skin S. The contact face 10 is substantially planar, in the sense that it extends parallel to a plane XY, although certain portions may be inclined relative to the plane XY. The main body also has a distal end 4, opposite the contact face 10. The contact surface 10 and the distal end 4 are connected to each other by a lateral face 5, extending around a central axis Δ, parallel to a transverse axis Z, the latter being perpendicular to the XY plane.

The contact face 10 can be formed by a membrane permeable to CO₂ or a plate with inlet openings 12 allowing CO₂ to pass through. The contact face may comprise a hydrophobic membrane to prevent water vapor diffusion through the device 1. The contact face can be heated to increase blood flow. This improves collection.

According to one possibility, the device also comprises a heating element, making it possible to bring the contact face 10, delimiting the measurement chamber 20, to a temperature higher than 37 °C, and preferably between 40 °C and 50 °C, and more preferably between 40 °C and 45 °C, for example 42 °C. The heating element is, for example, a resistor on the contact face, producing Joule-effect heating. A local and moderate increase in temperature, in the vicinity of the skin, promotes an increase in blood flow by dilating blood capillaries, which increases the diffusion of a gas of interest transcutaneously through the skin.

The contact face 10 opens onto a measurement chamber 20. The function of the measurement chamber is to estimate a CO₂ concentration of the gas mixture flowing through the main body 2, parallel or substantially parallel to the central axisΔ. To this end, the measurement chamber 20 comprises a gas sensor 23. Several types of sensor can be used for this purpose, for example optical sensors or electrochemical sensors, the latter being based on metal oxides (MOX sensors), or photoacoustic sensors. The inventors consider that the use an optical sensor is preferable, and more specifically an infra-red sensor. Such a sensor requires no special maintenance, is particularly compact and inexpensive. Moreover, such a sensor is highly specific for characterizing chemical bonds. It is suitable for detecting small molecules, such as carbon dioxide.

The gas sensor is of the NDIR (Non Dispersive Infra Red) type. This type of sensor comprises a source of infrared radiation 24, generally emitting in a spectral band between 1 μm and 20 μm. It also comprises at least one measurement photodetector 25, sensitive to infrared radiation. The principle is based on the attenuation, by the gas being analyzed, of the infrared radiation emitted by the source. The infrared source 24 and the measurement photodetector 25 form the gas sensor 23. The measurement photodetector 25 is, for example, a thermopile. A filter 26 placed in front of the thermopile determines the wavelength of the infrared radiation measured by the thermopile. Various gas sensor configurations are described below.

The gas sensor 23 is configured so that the transcutaneous gas of interest, in this case CO₂, propagates between the light source and the photodetector, parallel to the central axis Δ, or substantially parallel to the central axis Δ. Substantially parallel means parallel with an angular tolerance of less than ±30° or à±20° to the parallel.

The infrared source emits light propagating perpendicularly to the transverse axis Z. Preferably, the light emitted by the source is a sheet-like beam perpendicular to the transverse axis Z. Along the transverse axis Z, the beam thickness is preferably less than 1 cm, and more preferably less than 5 mm. Preferably, the light sheet extends over at least 50%, or even at least 80%, of the cross-section of the contact face 10, the latter being between a few cm² and 25 or 30 cm². The term cross section refers to an area perpendicular to the transverse axis Z (or to the central axisΔ). The low thickness of the beam reduces response time. The large cross-sectional area of the beam increases the amount of gas sampled, and therefore the sensitivity of the measurement.

In order to promote flow along the transverse axis Z through the measurement chamber 20, the contact face has a multitude of inlet openings 12, distributed over a cross-section of a few cm², for example between 5 and 25 cm². Alternatively, the contact face is a porous membrane, permeable to CO₂. The measurement chamber 20 opens onto a collection chamber 30. The interface between the measurement chamber 20 and the collection chamber 30 can be formed by one large collection opening, or by a plurality of collection openings 22, arranged in a collection plate 21 and distributed over a large cross-sectional area, similar to the inlet openings 12. Each collection opening 22 opens into a collection chamber 30.

The collection chamber 30 has side openings 34 in the side face 5 of the device 1. The side openings are configured to allow ambient air into the device 1. Ambient air is the air outside the device. The flow of ambient air through the side openings 34 is directed towards the central axisΔ. Each side opening 34 is preferably oriented perpendicularly to the central axis Δ, so that the ambient air enters the device 1, through each side opening 34, in a direction perpendicular, or substantially perpendicular, to the central axis. Δ

The intake of ambient air is achieved by a pump 41, arranged to apply a negative pressure to the collection chamber 30 relative to ambient pressure. The pump 41 may be located in an exhaust duct 40, adjacent to the collection chamber 30. In addition to admitting ambient air into the collection chamber, the pump allows the gas of interest to flow through the measurement chamber 10 to the collection chamber 20, through the collection openings 22. Preferably, the flow rate is adjusted so that the flow of the gas of interest through the measurement chamber 20 is laminar. The air flow rate into the collection chamber can be between 0.1 and 10 mL/min, for example, depending on the volume of the chamber.

In the collection chamber 30, ambient air, admitted through the side openings 34, mixes with the gas of interest, admitted through the collection openings 22. The discharge duct 40 is also placed under negative pressure by the pump 41, so that the air, mixed with the gas of interest, is discharged into the discharge duct, up to a discharge opening 42, forming the distal end 4 of the device. In the example shown in FIGS. 1A and 1B, the air flows through the discharge opening 42 around the central axisΔ. Alternatively, the air flows through the discharge opening 42 along a discharge axis which is secant to, and in particular perpendicular to, the central axis. Δ

In the example shown, the side openings are arranged on two opposite sides 5₁, 5₂ of the side wall. This promotes symmetrical airflow through the device. However, such a symmetrical arrangement of side openings is not necessary. The side openings 34 can be arranged on only one side of the side wall, in which case the discharge duct is arranged on an opposite side of the side wall.

Each side opening can be associated with a CO₂ filter, for example a filter containing lime, to trap CO₂ present in the ambient air.

The device includes a processing unit 50. The processing unit 50 comprises computing means, for example a microprocessor or microcontroller, on-board the device, being connected by a wired or wireless link to a cell phone or PC-type computer. The processing unit 50 is also configured to implement the process of estimating the dissolved CO₂ content of the user's blood, from the CO₂ concentration resulting from the gas sensor 23.

The general principles governing the estimation of dissolved CO₂ content in blood from the CO₂ concentration detected in the gas sensor have been described in WO2020/249466.

The method involves calculating the carbon dioxide concentration in the measurement chamber from the measurements taken by the two thermopiles, using models describing the attenuation of infrared radiation at the two wavelengths associated with each thermopile (Beer Lambert model, linear quadratic model). These models are described in WO2020/249466.

FIG. 1B shows the fluid flows within device 1. Transcutaneous CO₂ is admitted to the device via inlet openings 12 in the contact face 10, which open into the measurement chamber 20 (see arrow F₁). CO₂ propagates through the measurement chamber by diffusion, parallel to the central axis, the latter being parallel to the transverse axis Z. (see arrows F₂). Ambient air is admitted through lateral openings 34 in the side wall (see arrows F₃). The gas mixture comprising ambient air and transcutaneous CO₂ is produced in collection chamber 30. This mixture travels towards the discharge opening 42 (see arrows F₄) to exit the device body 2 (see arrows F₅).

An important aspect of the device is the use of the pump 41 to form a convection flow of air through the collection chamber 30, to evacuate the transcutaneous air/gas mixture of interest. This also accelerates air circulation in the collection chamber. The result is a pumping effect that accelerates the transport of CO₂ from blood to air, through the skin and the measurement chamber. The result is a reduction in the device's response time.

The measurement chamber 20 preferably includes a temperature sensor as well as a humidity sensor and a pressure sensor.

The temperature sensor converts the measured concentration into pressure according to the law of perfect gases. Using the law of perfect gases, the partial pressure of carbon dioxide

$${P}_{{CO}_{2}}^{mes} ( {T}^{mes} )$$

in the measurement chamber at temperature T^{meas}, is deduced:

$${P}_{{CO}_{2}}^{mes} ( {T}^{mes} ) = {C}_{{CO}_{2}}^{mes} . R . {T}^{mes} \qquad ( 1 )$$

Where

$${C}_{{CO}_{2}}^{mes}$$

is expressed in (mol/m³), R is the perfect gas constant (0.0623637 m³·mmHg·K⁻¹·mol⁻¹), T^{meas} in K and

$${P}_{{CO}_{2}}^{mes}$$

in mmHg.

The atmospheric pressure

$${P}_{air}^{mes}$$

sensor is configured to calculate the relative pressure

$${P}_{{CO}_{2}}^{mes rel}$$

expressed in ppm (parts per million):

$${P}_{{CO}_{2}}^{mes rel} = \frac{{P}_{{CO}_{2}}^{mes}}{{P}_{air}^{mes}} . 1 {0}^{6} \qquad ( 2 )$$

Where

$${P}_{{CO}_{2}}^{mes} and {P}_{air}^{mes}$$

are expressed in the same unit, here in mmHg.

The humidity sensor is used to calculate the concentration of water vapor in the measurement chamber. Water vapor is an important radiation attenuation factor. To take humidity into account, the relative humidity value RH^{mes} supplied by the humidity sensor must be converted into the water vapor concentration

$${C}_{{H}_{2} O}^{mes}$$

The relative humidity corresponds to the partial pressure of water in the measurement chamber, noted

$${P}_{{H}_{2} O}^{mes} ( {T}^{mes} ) ,$$

over the saturation vapor pressure at the temperature T^{mes} of the measurement chamber.

$${RH}^{mes} {=}^{Δ} \frac{{P}_{{H}_{2} O}^{mes}}{{VP}_{H 2 O} ( {T}^{mes} )} \text{.100} \% \qquad ( 3 )$$

The H₂O concentration in the measurement chamber can be obtained using the expression:

$${P}_{{H}_{2} O}^{mes} = \frac{{n}_{{H}_{2} O}^{mes}}{{V}^{mes}} {RT}^{mes} = {C}_{{H}_{2} O}^{mes} . R . {T}^{mes} \qquad ( 4 )$$

Combining the two previous equations, one may obtain:

$${C}_{{H}_{2} O}^{mes} = \frac{{RH}^{mes} . {VP}_{H 2 O} ( {T}^{mes} )}{100 \% R . {T}^{mes}} \qquad ( 5 )$$

where VP_{H2O}(T^{mes}) is the saturated water vapor pressure values. As a function of temperature, these values can be obtained from tables, or from an analytical expression, such as the Tetens equation.

$${VP}_{H 2 O} ( {T}^{mes} ) = 0.61078 {e}^{( \frac{17.27 ( {T}^{mes} - 273.15 )}{{T}^{mes} - 35.85} )} \qquad ( 6 )$$

The

$${C}_{{H}_{2} O}^{mes}$$

concentration can be used to estimate the CO₂ concentration in the measurement chamber, using the Beer-Lambert expression

$$- ln [ \frac{U ( {λ}_{1} ) / U ( {λ}_{2} )}{{U}_{0} ( {λ}_{1} ) / {U}_{0} ( {λ}_{2} )} ] = [ {k}_{{CO}_{2}} ( {λ}_{1} ) - {k}_{{CO}_{2}} ( {λ}_{2} ) ] . {C}_{{CO}_{2}} + [ {k}_{{H}_{2} O} ( {λ}_{1} ) - {k}_{{H}_{2} O} ( {λ}_{2} ) ] + [ {A}_{air} ( {λ}_{1} ) - {A}_{air} ( {λ}_{2} ) ] \qquad ( 7 )$$

Where C_{CO}_{₂} and C_{H}_{₂}_{O} are the molar concentrations, k_{CO}_{₂}(λ₁), k_{CO}_{₂}(λ₂), k_{H}_{₂}_{O}(λ₁), k_{H}_{₂}_{O}(λ₂) are the attenuation coefficients of carbon dioxide and water vapor, Aₐᵢᵣ(λ₁)Aₐᵢᵣ(λ₂) are the attenuations of air at wavelengths λ₁, λ₂

Taking H₂O concentration into account improves the accuracy with which CO₂ concentration is measured, as mentioned in patent application WO2020/249466.

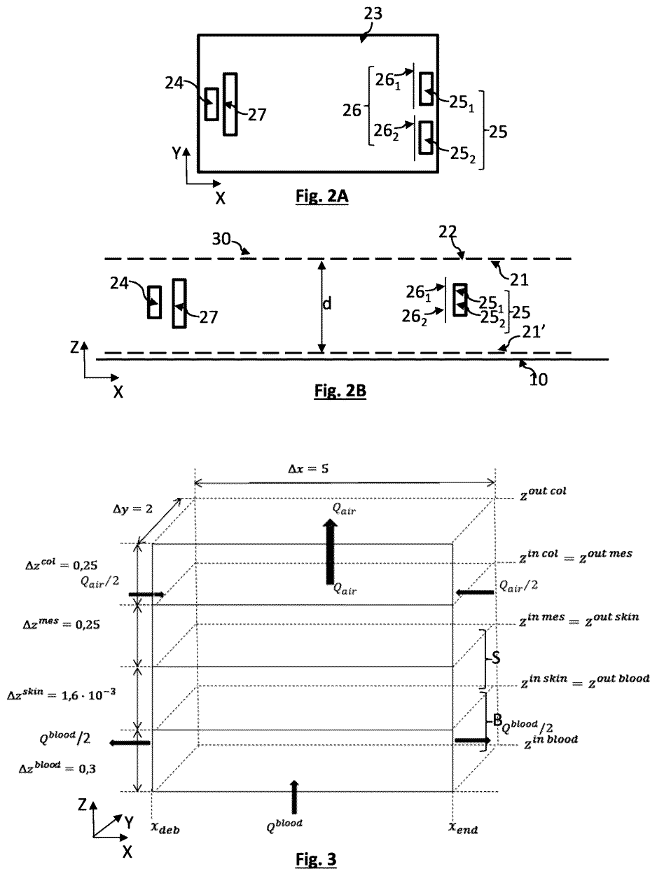

FIG. 2B shows the measurement chamber in a plane P_{XZ} parallel to the transverse axis Z. The measurement chamber may extend between two grids 21 and 21′ permeable to the gas of interest. Grid 21 forms the interface wall with the collection chamber 30, and comprises collection apertures, allowing the gas of interest to flow from the measurement chamber into the collection chamber. Preferably, each grid is reflective in the spectral emission band of the infrared source. The measurement chamber may include a reflective grid 21′, similar to grid 21, at the contact face 10. The presence of each reflective grid increases the amount of light propagating through the gas of interest to each channel of the photodetector. It also limits the thickness of the light sheet along the transverse axis.

The distance d between grid 21 and the contact face, or between grids 21 and 21′, is, for example, between 3 mm and 8 mm.

The measurements resulting from the sensor may be noisy, in which case low-pass filtering can be applied. Low-pass filtering can be achieved, for example, by a moving-average filter whose length is adapted to the level of measurement noise.

From

$${C}_{CO 2}^{mes} ,$$

the partial pressure CO2

$${P}_{CO 2}^{mes} ( {T}^{mes} )$$

is deduced by:

$${P}_{CO 2}^{mes} ( {T}^{mes} ) = {C}_{CO 2}^{mes} . {RT}^{mes} \qquad ( 8 )$$

From the atmospheric pressure sensor in the measurement chamber, the relative pressure

$${P}_{CO 2}^{mes , rel} ( {T}^{mes} )$$

is calculated by:

$${P}_{CO 2}^{mes , rel} ( {T}^{mes} ) = \frac{{P}_{CO 2}^{mes} ( {T}^{mes} )}{{P}_{air}^{mes}} {10}^{6} \qquad ( 9 )$$

Where

$${P}_{air}^{mes}$$

is the atmospheric pressure in the measurement chamber.

From the CO2 concentration resulting from the measurement sensor, the carbon dioxide concentration in the blood can be estimated by applying a model of CO2 transport between the blood and the measurement chamber. A monodirectional transport model is described below, modeling CO2 transport by diffusion and convection.

### First Model: One-Dimensional 1D Model

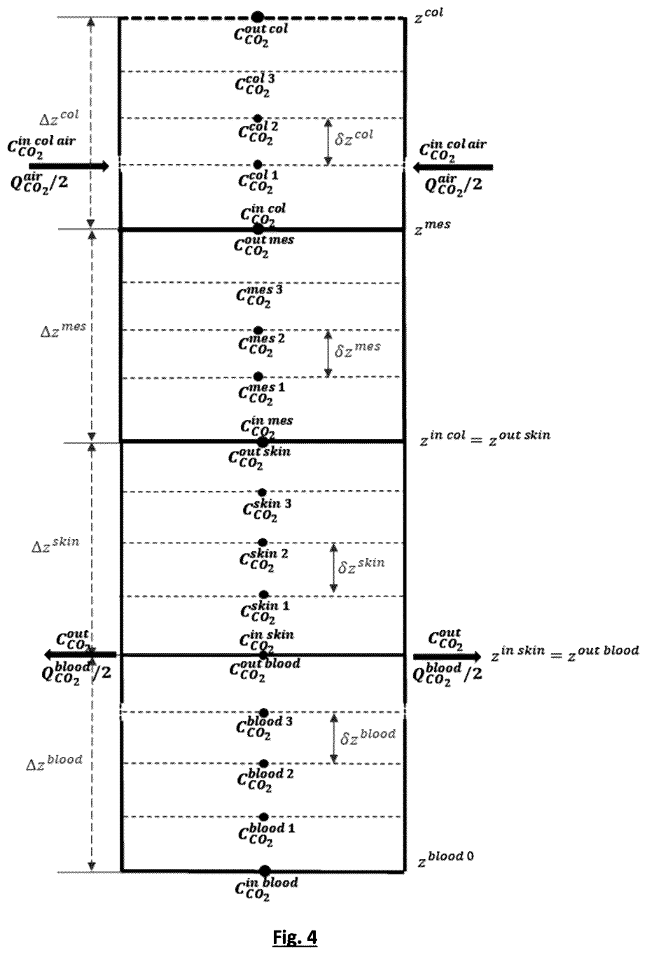

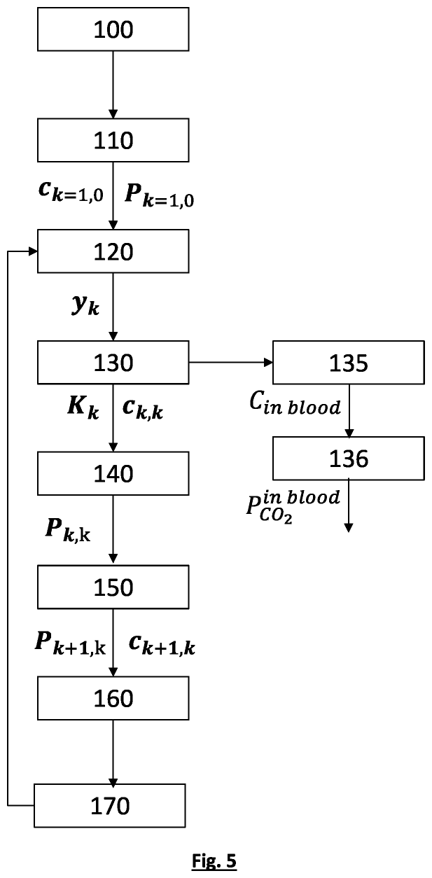

FIG. 3 shows an example of a device applied to the user's skin S. The blood occupies a blood compartment B, between the coordinates z^{in blood} and z^{out blood}, and its thickness is Δz^{blood}.

Blood compartment B is covered by skin S. Skin S extends between coordinates z^{in skin} and z^{out skin}, and its thickness is. Δz^{skin}. z^{in skin}=z^{out blood} The skin is covered by the device. In the prior art, the device comprises a superposition of the collection chamber and the measurement chamber. The collection chamber extends between the coordinates z^{in col} and z^{out col}, and its thickness is Δz^{col}. The measurement chamber extends between the coordinates z^{in mes} and z^{out mes}, and its thickness is Δz^{mes}. z^{in col}=z^{out peau}; z^{in mes}=z^{out col}. According to the invention, the device comprises a superposition of the measurement chamber 20 and the collection chamber 30.

Diffusion is characterized by a diffusion coefficient, which depends on the medium through which it passes, in this case blood, skin and air.

In 1D modeling, carbon dioxide transport is described only along the central axis Δ corresponding to the Z axis. In the blood compartment, blood flows in a convection current with mean velocity

$${u}_{z}^{blood} .$$

In the skin, CO₂ migrates solely by diffusion. In the device, in the measurement chamber CO₂ migrates solely by diffusion, and in the collection chamber CO₂ migrates by diffusion and convection. Convection results from pump-induced air circulation in the collection chamber. Air convection in the collection chamber leads to diffusion across the contact face and through the measurement chamber. It is characterized by an average velocity

$${u}_{z}^{col}$$

along the Z axis.

Based on propagation of the gas of interest in a direction Z perpendicular to the skin surface (one-dimensional model), in any homogeneous medium (i-e of the same Henry's law constant), one can write:

$$\frac{\partial C ( z , t )}{\partial t} = \frac{\partial }{\partial z} [ - u ( z ) C ( t , z ) ] + \frac{\partial }{\partial z} [ D ( z ) \frac{\partial C ( t , z )}{\partial z} ] + R ( z , t ) \qquad ( 10 )$$

where:
u(z) is the propagation speed of CO2 at a coordinate z along the Z axis;
   C(t, z) is the CO2 concentration at time t and coordinate z;
   D(z) is the diffusion coefficient of CO2 along the Z propagation direction at the z coordinate.
   R(z, t) corresponds to the outflow of CO2 into the blood at skin level (sink term) and the inflow of CO2 through the side walls into the collection chamber (source term). R(z, t) is therefore non-zero only at the blood/skin interface and the measurement chamber/collection chamber interface inside the device.

Considering a homogeneous medium:

$$\frac{\partial }{\partial z} D ( z ) = 0 et \frac{\partial }{\partial z} u ( z ) = 0$$

Equation (10) becomes

$$\frac{\partial C ( z , t )}{\partial t} = - {u}_{z} \frac{\partial C ( z , t )}{\partial z} + D \frac{{\partial }^{2} C ( z , t )}{\partial {z}^{2}} + R ( z , t ) \qquad ( 11 )$$

In expression (11), the

$$- {u}_{z} \frac{\partial C ( z , t )}{\partial z}$$

term corresponds to the dynamics due to convection. The term

$$D \frac{{\partial }^{2} C ( z , t )}{\partial {z}^{2}}$$

corresponds to dynamics due to diffusion. Expression (11) corresponds to a convection-diffusion equation.

Expression (11) models the transport of the gas of interest along the Z axis within each compartment described above (measurement chamber, collection chamber).

The model also takes into account an observation equation:

C(zₘₑₛ, t)=y (12) where y is the measured concentration

$${C}_{{CO}_{2}}^{mes}$$

in the measurement cell and zₘₑₛ is the Z-axis spatial coordinate of the measurement point.

The boundary conditions are:

C(z_{blood 0},t)=C_{in blood}(t) (12′)
where z_{blood 0} corresponds to the Z coordinate of the blood compartment inlet. and

$$\frac{\partial C ( {z}_{col} , t )}{\partial z} = 0 \qquad ( 13 )$$

where z_{col} is the Z coordinate of the distal end of the device.

The conditions at the interfaces are now described. In the following equations, indices i and j designate two successive media respectively upstream and downstream of the interface, upstream and downstream being taken into account in the direction of CO2 propagation, i.e. from the blood compartment to the collection chamber.

Considering that z_{i|j} represents the spatial coordinate along the Z axis of the interface between medium i and medium j, Jᵢ(z_{i|j}, t) is the gas flow exiting medium i and Jⱼ(z_{i|j}, t) is the gas flow entering medium j, per unit area. For a gas-transparent interface:

Jᵢ(z_{i|j},t)=Jⱼ(z_{i|j},t) (15)

with:

Jᵢ(z_{i|j},t)=uᵢ(z_{i|j})C(z_{i|j},t)−Dᵢ(z_{i|j})∇C(z_{i|j},t) (16)

and

Jⱼ(z_{i|j},t)=uⱼ(z_{i|j})C(z_{i|j},t)−Dⱼ(z_{i|j})∇C(z_{i|j},t) (17)

In expressions (16) and (17), uᵢ(z_{i|j})C(z_{i|j}, t) and uⱼ(z_{i|j})C(z_{i|j}, t) correspond to convection, while Dᵢ(z_{i|j})∇C(z_{i|j}, t) and Dⱼ(z_{i|j})∇C(z_{i|j}, t) correspond to diffusion.

At the interfaces between blood and skin, or skin and measurement chamber, or measurement chamber and collection chamber, the velocity is assumed to be zero on either side of the interface: uᵢ(z_{i|j})=uᵢ(z_{i|j})=0. In fact, convection only takes place in the collection chamber. In the skin and in the measurement chamber, CO2 propagates solely by diffusion.

Combining (16) and (17), due to the continuity of the flow, and introducing a transparency factor, one my obtain:

$${r}_{transpa i \vert j} {D}_{i} \frac{\partial {C}_{i} ( {z}_{i \vert j} , t )}{\partial z} = {D}_{j} \frac{\partial {C}_{j} ( {z}_{i \vert j} , t )}{\partial z} \qquad ( 18 )$$

Where τ_{transpa i|j} corresponds to the transparency factor between compartment i and compartment j. The transparency factor corresponds to the ratio between the open surface area of the interface, through which the gas of interest passes, and the total surface area of the interface.

The pressure on either side of the interface is identical:

Pᵢ(z_{i|j},t)=Pⱼ(z_{i|j},t) (19)

However

$${C}_{i} ( {z}_{i \vert j} , t ) = \frac{{H}_{i}}{{RT}_{i}} {P}_{i} ( {z}_{i \vert j} , t ) \qquad ( 20 )$$

Where Tᵢ is the temperature of medium i, and Tᵢ=Tⱼ

$$\frac{{C}_{i} ( {z}_{i \vert j} , t )}{{H}_{i}} = \frac{{C}_{j} ( {z}_{i \vert j} , t )}{{H}_{j}} {=}^{def} {C}_{i \vert j} ( {z}_{i \vert j} , t ) \qquad ( 21 )$$

The symbol def means [symbol] means “equality that serves as a definition”.

### Consideration of Blood Flow in the Blood Compartment

In blood compartment B:

$$R ( {z}^{blood 3} , t ) = - \frac{2 {u}_{x}^{blood}}{Δ x} {C}^{out} \qquad ( 22 )$$

Δx is the width of the input window
C^{out} is the concentration of CO₂ leaving the blood compartment as a result of blood flow;

$$- {u}_{x}^{blood}$$

is the blood flow velocity in a direction perpendicular to the Z axis.
R(z^{blood 3}, t) corresponds to a sink term.

$${u}_{x}^{blood} = \frac{\frac{{Q}^{blood}}{2}}{{A}_{x}^{out blood}} \qquad ( 23 )$$

Q^{blood} is the blood flow rate and

$${A}_{x}^{out blood}$$

is the outlet area to the left and right of the blood compartment. This is a cross-section of the blood vessels.
Consideration of Air Intake into the Collection Chamber.

In the collection chamber, air intake results in a source term corresponding to the carbon dioxide present in the ambient air.

$$R ( {z}^{col 1} , t ) = \frac{2 {u}_{x}^{col}}{Δ x} {C}^{in col air} \qquad ( 24 )$$

Δx corresponds to the width of the device

$$- {u}_{x}^{col}$$

is the air intake current
C^{in col air} is the concentration of air entering the collection chamber;
R(z^{col 1}, t) corresponds to a source term.

$${u}_{x}^{col} = \frac{\frac{{Q}^{air}}{2}}{{A}_{x}^{in col}} \qquad ( 25 )$$

Q^{air} is the air flow rate and

$${A}_{x}^{in col}$$

side walls of the collection cell.

### Model Discretization.

The device and the environment on which it is applied have been discretized.

The convection-diffusion equation (11) can be discretized spatially, as shown in FIG. 4. A spatial mesh has been created, generating 17 equally spaced sampling points in each compartment, between the blood compartment B and the collection chamber. Each compartment has 5 sampling points, 2 of which are located at a lower or upper interface. At an interface between a downstream and an upstream compartment, the concentration leaving the downstream compartment is referenced by an “out” superscript, while the concentration entering the upstream compartment is referenced by an “in” superscript.

The concentration within the blood compartment is discretized according to 3 sampling points:

$${C}_{CO 2}^{blood , 1} ; {C}_{CO 2}^{blood , 2} ; {C}_{CO 2}^{blood , 3}$$

The concentration inside the skin compartment is discretized according to 3 sampling points:

$${C}_{CO 2}^{skin , 1} ; {C}_{CO 2}^{skin , 2} ; {C}_{CO 2}^{skin , 3}$$

The concentration inside the measurement chamber is discretized according to 3 sampling points:

$${C}_{CO 2}^{mes , 1} ; {C}_{CO 2}^{mes , 2} ; {C}_{CO 2}^{mes , 3}$$

Concentration in and out of the collection chamber is discretized according to 4 sampling points:

$${C}_{CO 2}^{col , 1} ; {C}_{CO 2}^{col , 2} ; {C}_{CO 2}^{col , 3} ; {C}_{CO 2}^{out col} .$$

At the interfaces between compartments, concentrations are

CO2 concentrations at the interfaces can be defined:
At the blood/skin interface:

$$\frac{{C}_{CO 2}^{out blood}}{{H}^{blood}} = \frac{{C}_{CO 2}^{in skin}}{{H}^{skin}} ;$$

H^{blood} and H^{skin} correspond to the Henry constants of blood and skin.

$$\frac{{C}_{CO 2}^{out skin}}{{H}^{skin}} = \frac{{C}_{CO 2}^{in mes}}{{H}^{air}}$$

H^{air} corresponds to the Henry's law constant of air.
At the measuring cell/collection cell interface

$${C}_{CO 2}^{out mes} = {C}_{CO 2}^{in skin}$$

The input concentration is

$${C}_{CO 2}^{in blood} .$$

In the direct problem formulation,

$${C}_{CO 2}^{in blood}$$

is known. When solving the inverse problem,

$${C}_{CO 2}^{in blood}$$

is sought.

The spatial distance δz between each mesh point in the same compartment is identical. Hereafter, the spacing between each mesh point is noted δzᵢ, the index i designating the medium: i=1 for blood, i=2 for skin, i=3 for the measurement chamber and i=4 for the collection chamber.

Given the spatial discretization, the convection-diffusion equation (expression 11) becomes:

$$\frac{C ( z , t + δ t ) - C ( z , t )}{δ t} = [ \frac{u δ {z}_{i} + 2 D}{2 {( δ {z}_{i} )}^{2}} ] C ( z - δ {z}_{i} , τ ) + [ \frac{- 2 D}{{( δ {z}_{i} )}^{2}} ] C ( z , τ ) + [ \frac{- u δ {z}_{i} + 2 D}{2 {( δ {z}_{i} )}^{2}} ] C ( z + δ {z}_{i} τ ) \qquad ( 30 )$$

In this example, τ=t+δt, an implicit integration scheme is used.

A vector z is defined, of dimension (17, 1), which contains all the mesh points.

$$z = {( {z}_{i} )}_{i = 1}^{n} \qquad ( 31 )$$

The total number of samples is N, such that

$$N = {\sum }_{i = 1}^{4} {N}_{i} \qquad ( 32 )$$

where Nᵢ is the number of samples in the compartment. i

$${N}_{i} = \frac{Δ {z}_{i}}{δ {z}_{i}} - 1$$

where Δzᵢ is the height of the medium i and the distance between two successive points in the medium i is noted δzᵢ

The position, along z, in the column, at which each medium begins, Is defined by z_{deb i}. This position can be calculated in general terms using the following relationships:

$${z}_{deb i} = \{ \begin{matrix}{z}_{0} , with i = 1 \\ {z}_{0} + {\sum }_{j = 1}^{i = 1} Δ {z}_{i} , with i \geq 2\end{matrix} \qquad ( {32}^{′} )$$

### Direct Model

A vectorc is defined in relation to the concentrations assessed at various sampling points defined by the vector z.

c₀ is a vector of dimension (17, 1). It is obtained by concatenating the terms of the vectors

$${c}_{i}^{i} ( t )$$

with i between 1 and. 4

$${c}_{0} = [ \begin{matrix}c ( {z}_{deb 1} + k . δ {z}_{1} ) , & k = 1 , … , {N}_{1} \\ c ( {z}_{deb 2} + k . δ {k}_{2} ) , & k = 1 , … , {N}_{2} \\ c ( {z}_{deb 3} + k . δ {k}_{3} ) , & k = 1 , … , {N}_{3} \\ c ( {z}_{deb 4} + k . δ {z}_{4} ) , & k = 1 , … , ( {N}_{4} + 1 )\end{matrix} ] \qquad ( 34 )$$

In this example, we assume that CO2 is measured at the outlet of the measurement chamber

y=C(z_{deb 3}+N₃δz₃)

Nₛₜₐₜₑ groups points at the upper edge and strictly inside the media, excluding the 3 points corresponding to the blood/skin, skin measurement and measurement/collection interfaces

Nₛₜₐₜₑ=N−3

In this case: N₁=N₂=N₃=N₄=3 so N=16 and Nₛₜₐₜₑ=13

A vectorc Is defi relation to the concentrations evaluated at the various state points.

An augmented state vector \tilde{c} of dimension (Nₛₜₐₜₑ+1), is defined by adding the concentration C^{in blood} at the blood medium Inlet, to the vector c:

Expression (30) can be written as a matrix:

c(t+δt)−c(t)=δt·P·c(τ) (36)

c(t) and c(t+δt) are vectors of dimension (13, 1). Each term in these vectors corresponds to concentration at a measurement point shown in FIG. 4, at time t and time t+δt respectively,
F is a transfer matrix of dimension (13,13), such that

F=F_{conv}+F_{dif}+Fᵢₙₜ+Fₚᵤᵢₜ (37)

Where:
F_{conv} groups convection-related discretizations: F_{conv} of dimension [Nₛₜₐₜₑ, Nₛₜₐₜₑ] (Nₛₜₐₜₑ=13 this example), is calculated as follows on terms of rank (q, q+1)

$${f}_{q , q + 1} = - \frac{{u}_{i}}{2 δ {z}_{i}} \qquad ( 38 )$$

and on terms of ranks (q,q−1)

$${f}_{q , q - 1} = - \frac{{u}_{i}}{2 δ {z}_{i}} \qquad ( 39 )$$

Apart from the points of rank (q,q+1) and (q, q−1), the terms of F_{conv} have zero value.
F_{dif} groups the discretizations related to diffusion;
Fᵢₙₜ groups interface and edge discretizations;
An Fₚᵤᵢₜ contains the sink term (i.e. the exit of blood from the skin Into the bloodstream);
uₗ is the propagation velocity in the z direction for the compartment i and δzᵢ is the distance between two adjacent sampling points in compartment i
F_{dif} of dimension [Nₛₜₐₜₑ, Nₛₜₐₜₑ] (Nₛₜₐₜₑ=13 In this example) is calculated as follows:
   on the main diagonal (row and column of the same rank q);

$${f}_{q , q} = - \frac{- 2 {D}_{i}}{{( δ {z}_{i} )}^{2}} \qquad ( 40 )$$

above the main diagonal

$${f}_{q , q + 1} = \frac{{D}_{i}}{{( δ {z}_{i} )}^{2}} \qquad ( 41 )$$

below the main diagonal

$${f}_{q , q - 1} = \frac{{D}_{i}}{{( δ {z}_{i} )}^{2}} \qquad ( 42 )$$

Apart from the points of rank (q, q+1) and (q, q−1), (q,q); the terms of F_{dif} zero value. The matrix Fᵢₙₜ, describing the conditions at the interfaces between two adjacent compartments, of dimension [Nₛₜₐₜₑ,Nₛₜₐₜₑ] (Nₛₜₐₜₑ=13 in this example), is calculated as follows:

All terms of the matrix Fᵢₙₜ are zero,
The terms [1,1] and [1,2], which correspond to the lower edge, at the coordinate z^{blood 1}=z_{deb1}+1
The terms [(z_{deb1}+N₁·δz₁, z_{deb1}+N₁·δz₁),(z_{deb1}+N₁·δz₁, z_{deb2}+δz₂)] and [(z_{deb2}+δz₂, z_{deb1}+N₁·δz₁),(z_{deb2}+δz₂, z_{deb2}+δz₂)] which correspond to the first interface;
The terms [(z_{deb2}+N₂·δz₂, z_{deb2}+N₂·δz₂),(z_{deb2}+N₂·δz₂, z_{deb3}+δz₃)] and [(z_{deb3}+δz₃, z_{deb2}+N₂·δz₂),(z_{deb3}+δz₃, z_{deb3}+δz₃)] which correspond to the second Interface;
The terms [(z_{deb3}+N₃·δz₃, z_{deb3}+N₃·δz₃),(z_{deb3}+N₃·δz₃, z_{deb4}+δz₄)] and [(z_{deb4}+δz₄, z_{deb3}+N₃·δz₃),(z_{deb4}+δz₄, z_{deb4}+δz₄)] which correspond to the third interface;
The terms (z_{deb4}+(N₄+1)·δz₄, z_{deb4}+N₄·δz₄), (z_{deb4}+(N₄+1)·δz₄, z_{deb4}+(N₄+1)·δz₄) are equal and correspond to the top edge.

Matrix terms other than those explained above are equal to 0.

Each non-zero term f_{q,q} of the matrix Fᵢₙₜ is such that:

$${f}_{q , q} = - \frac{{u}_{i} δ {z}_{i} + 2 {D}_{i}}{2 {( δ {z}_{i} )}^{2}} \qquad ( 43 )$$

With [q, q]=[z_{deb1}+N₁·δz₁, z_{deb1}+N₁·δz₁], [z_{deb2}+N₂·δz₂, z_{deb2}+N₂·δz₂], [z_{deb3}+N₃·δz₃, z_{deb3}+N₃·δz₃]

Each non-zero term f_{q,q} of the matrix Fᵢₙₜ is such that:

$${f}_{q , q + 2} = {λ}_{i \vert j} \frac{{D}_{j}}{δ {z}_{j}} \qquad ( 44 )$$

with [q,q+2]=[z_{deb1}+N₁·δz₁, z_{deb2}+δz₂], [z_{deb2}+N₂·δz₂, z_{deb3}+δz₃], [z_{deb3}+N₃·δz₃, z_{deb4}+δz₄]
and

$${λ}_{j \vert i} = [ \frac{{u}_{j} δ {z}_{j} + 2 {D}_{j}}{2 {( δ {z}_{j} )}^{2}} ] {H}_{j} {ζ}_{i \vert j} \qquad ( 45 )$$

$$and$$

$${ζ}_{i \vert j} = \frac{1}{{r}_{transpa i \vert j} {H}_{i} \frac{{D}_{i}}{δ {z}_{i}} + {H}_{j} \frac{{D}_{j}}{δ {z}_{j}}} \qquad ( 46 )$$

Hⱼ and Hᵢ are the respective Henry coefficients of the media i and j and r_{transpa i|j} is the transparency factor between media i and j.

The matrix Fₚᵤᵢₜ, describing the conditions at the interfaces between two adjacent compartments, of dimension [Nₛₜₐₜₑ,Nₛₜₐₜₑ] (Nₛₜₐₜₑ=13 in this example), is calculated as follows. The only non-zero term is the term located at position (3, 2) corresponding to the interface between blood and skin

$${F}_{puit} = ( \begin{matrix} & \begin{matrix}0 & … & 0 \\ ⋮ & … & ⋮\end{matrix} & \\ 0 & \frac{- {u}_{z}^{blood}}{2 δ {z}^{blood}} & 0 \\ & \begin{matrix}⋮ & … & ⋮ \\ 0 & … & 0\end{matrix} & \end{matrix} ) \qquad ( 47 )$$

The

$$\frac{- {u}_{z}^{blood}}{2 δ {z}^{blood}}$$

term corresponds to the convection outlet from the blood compartment.

The air supply to the collection cell is taken into account in the control matrix described below.

### Application of a Recursive Estimator.

In this example, a recursive estimator is used to estimate the concentration of CO2 in blood as a function of time. This is a linear recursive estimator of the Kalman filter type.

The estimator is based on the spatio-temporal state-space model described above, to which an observation equation is added, translating the relationship between each measurement and the states.

As known with this type of estimator, the concentration at an instant is calculated recursively, from an estimate of said concentration, resulting from a previous iteration, and from a measurement taken at instant t.

Starting from equation (36):

C(z,t+δt)−C(z,t)=δt·F·C(z,t+δt);

thus:

$${\underbrace{( I - δ t \cdot F )}}_{A} C ( z , t + δ t ) = C ( z , t ) \qquad ( 50 )$$

In the following, the following notations are used:
\tilde{c}_{k,k-1} state vector at time k predicted at previous time k−1: dimension (Nₛₜₐₜₑ+1);
\tilde{c}_{k,k} state vector at time k calculated at time k: dimension (Nₛₜₐₜₑ+1);
qₖ control vector, dimension (Q, 1). In this example, Q=1.
\tilde{c}_{k+1,k} state vector at time k+1 predicted at time k: dimension (Nₛₜₐₜₑ+1);
P_{k,k-1} covariance matrix of the error at time k predicted at the previous time k−1: dimension (Nₛₜₐₜₑ+1,Nₛₜₐₜₑ+1);
P_{k,k} the covariance matrix of the error at time k calculated at time k: dimension
(Nₛₜₐₜₑ+1,Nₛₜₐₜₑ+1);
P_{k+1,k} covariance matrix of the error at time k+1 predicted at time k: dimension (Nₛₜₐₜₑ+1,Nₛₜₐₜₑ+1)
Kₖ Kalman gain matrix calculated time k: dimension (Nₛₜₐₜₑ+1, M)
yₖ measurement observed at time k: dimension (M). In this example, the measurement is a scalar: M=1.
H̅: observation matrix: dimension (Nₛₜₐₜₑ+1,M);
G: control matrix: dimension (Nₛₜₐₜₑ+1, Q)
\tilde{w}ₖ: modeling noise: dimension (Nₛₜₐₜₑ+1);
vₖ: observation noise: dimension (M); Observation and modeling noise are assumed to be white and independent;
Rₖ: observation noise covariance matrix at time A. Dimension (M, M);
Qₖ: system noise covariance matrix at time k. Dimension (Nₛₜₐₜₑ+1,Nₛₜₐₜₑ+1);
\tilde{F}: enhanced transition matrix. Dimension (Nₛₜₐₜₑ+1, Nₛₜₐₜₑ+1).

$$w [ k ] \sim 𝒩 ( 0 , {σ}_{w}^{2} )$$

is the noise that incorporates model errors, and

$$v [ k ] \sim 𝒩 ( 0 , {σ}_{v}^{2} )$$

describes the noise on the values provided by the sensor, Their covariance matrices are defined:

$$𝔼 [ {w}_{k} {w}_{n}^{T} ] = \{ \begin{matrix}{Q}_{k} , k \neq n \\ 0 , k = n\end{matrix} 𝔼 [ {v}_{k} {v}_{n}^{T} ] = \{ \begin{matrix}{R}_{k} , k \neq n \\ 0 , k = n\end{matrix} \qquad ( 51 )$$

$${σ}_{w}^{2} .$$

means a normal distribution with mean zero and variance

$$𝒩 ( 0 , {σ}_{w}^{2} )$$

~ means: is the distributed according to
[symbol] is the expectation operator
w[k] and v[k] are assumed to be mutually independent.

$$𝔼 [ {w}_{k} {v}_{n}^{T} ] = 0 \qquad ( 52 )$$

The process comprises an Initialization phase, during which an initial state vector c_{k=1,0} a and a covariance matrix of the error at the initial time P_{k=1,0} are used.

The correction step is defined by e following equations:

The Kalman is estimated by

Kₖ=P_{k,k-1}H̅^{T}(\tilde{H}P_{k,k-1}\tilde{H}^{T}+Rₖ)⁻¹ (60)

The Kalman gain represents the weights (i.e. the confidence) given to the noisy measurement yₖ and to its estimate H̅c̅_{k,k-1}.

Estimation of \tilde{c}_{k,k} concentration at time k:

\tilde{c}_{k,k}=\tilde{c}_{k,k-1}+Kₖ(yₖ−\tilde{H}x_{k,k-1}) (61)

Estimation of the covariance matrix of the estimation error P_{k,k}:

$${P}_{k , k} = ( I - {K}_{k} \tilde{H} ) {{P}_{k , k - 1} ( I - {K}_{k} \tilde{H} )}^{T} + {K}_{k} {R}_{k} {K}_{k}^{T} \qquad ( 62 )$$

The estimation of \tilde{c}_{k,k} allows to know C_{in blood}, the latter being the rank 1 term of the vector \tilde{c}_{k,k}. From C_{in blood},

$${P}_{{CO}_{2}}^{in blood}$$

can be estimated.

The prediction step is defined by the following equations:

Concentration prediction at time k+1:

$$\tilde{A} {\tilde{c}}_{k + 1 , k} = {\tilde{c}}_{k , k} + \tilde{G} {C}_{k + 1}^{in col air} \qquad ( {62}^{′} )$$

Prediction of the error covariance matrix P_{k+1,k}

P_{k+1,k}=Ã⁻¹P_{k,k}Ã⁻¹^{^{T}}+\tilde{Q} (63)

The variable to be estimated, C_{in blood}, is found in the state vector \tilde{c}ₖ at position z_{deb1} (rank 1 term). It is linked to the following state variable C_{blood1} by the terms of the transport equation. The associated equation of state is:

C_{in blood}[k+1]=φC_{in blood}[k]+w[k+1] (64)

Where φ is a parameter characterizing an a priori on the signal C_{in blood}. If the a priori is that the input signal is stationary, then φ=1

Expression (50) can be expressed by:

Ã\tilde{c}ₖ₊₁=\tilde{c}ₖ+\tilde{G}qₖ₊₁+\tilde{w}ₖ₊₁ (65)

where
\tilde{c}ₖ is the previously defined augmented state vector \tilde{c}k=\tilde{c}(tₖ). The augmented state vector includes the concentration in each compartment as well as the concentration in blood. For an implicit time integration scheme, one can define:

Ã=I−\tilde{F}·δt (66)

Where the augmented transition matrix \tilde{F} is
F_{[(N+1)×(N+1)]} is the matrix defined by:

$$F = [ \begin{matrix}φ & 0 \\ {g}_{11} & \\ 0 & \\ 0 & [ F ] \\ 0 & \\ 0 & \end{matrix} ]$$

$$\overline{G} = [ \begin{matrix}0 \\ \overline{0} \\ g \\ 0 \\ \overline{0}\end{matrix} ]$$

Where

$$g = 2 \frac{{u}_{x}^{col}}{Δ x}$$

for the term defined by the z axis z_{deb 4}+δz₄ and
\tilde{w} is the vector containing the state noise. It has the dimension (Nₛₜₐₜₑ+1)×1

$$w = ( \begin{matrix}{w}^{in blood} \\ \\ … \\ w\end{matrix} )$$

For an Implicit time integration scheme, the discrete equation of state is written:

Ã\tilde{c}[k+1]=\tilde{c}[k]+\tilde{G}C^{in col air}[k+1]+\tilde{w}[k+1]

w^{in blood} follows a normal distribution

$$𝒩 ( 0 , {σ}_{w}^{2} )$$

and

$${q}_{k + 1} = ( {C}_{{CO}_{z}}^{in col air} ( {z}^{col 1} , k + 1 ) )$$

Where the matrix Ã of dimension (Nₛₜₐₜₑ+1)×(Nₛₜₐₜₑ+1) is defined by:

Ã=1−\tilde{F}·δt

Where I is the ide dimension (Nₛₜₐₜₑ+1)×(Nₛₜₐₜₑ+1)

The observation equation for the augmented system is:

yₖ=\tilde{h}^{t}\tilde{c}ₖ+vₖ
yₖ is the observation noised from the actual concentration value

$${\overline{c}}_{k} , {y}_{k} = {C}_{{CO}_{2}}^{mes 3}$$

$$v [ k ] ∼ 𝒩 ( 0 , {σ}_{v}^{2} )$$

describes the observation noise on the values provided by the sensor
\tilde{h} is the augmented observation

$$\cdot v [ k ] \sim 𝒩 ( 0 , {σ}_{v}^{2} )$$

In this case, since observation is defined by a single value, the augmented observation matrix \tilde{H} is reduced to an observation vector. \tilde{h}

yₖ=\tilde{H}^{t}\tilde{c}ₖ+vₖ

where:

\tilde{H}=\tilde{h}

The system of equations is

$$\{ \begin{matrix}\tilde{A} {\tilde{c}}_{k + 1} = {\tilde{c}}_{k} + \tilde{G} {C}_{k + 1}^{in col air} + {\tilde{w}}_{k + 1} \\ {y}_{k} = {\tilde{H}}^{t} {\tilde{c}}_{k} + {v}_{k}\end{matrix}$$

The Kalman algorithm recursively links a step of correction of the state vector at an instant k (or tₖ) and prediction of the state vector

From C_{in blood}, the CO2 content of the blood can be

$${P}_{{CO}_{2}}^{in blood} = \frac{{C}_{in blood}}{{β}^{blood}} \qquad ( 67 )$$

β^{blood} is the Ostwald solubility coefficient of carbon dioxide in blood.

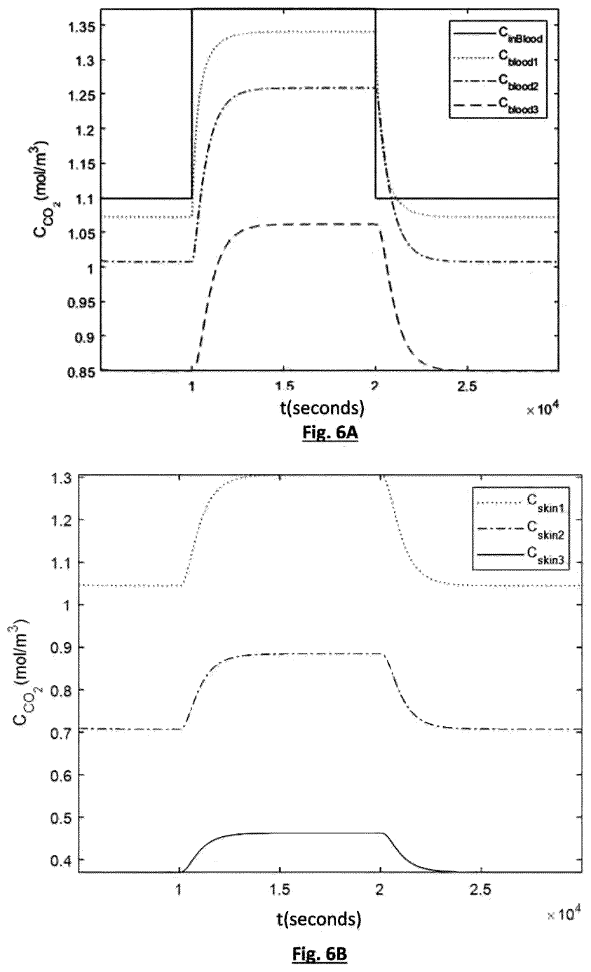

FIG. 5 shows the main steps involved in implementing a process according to the invention.

Step 100: Placing the measuring device against the medium being analyzed and activate the pump.

Step 110: Initialization: In this step, an initial state vector c_{k=1,0} and an error covariance matrix P_{k=1,0} are defined. The measurement instant k is then incremented: k=2.

Step 120: Measurement of CO₂ concentration in the measurement chamber. The measured variable yₖ is thus obtained.

Step 130: From the initial state vector c_{k=1,0}, or the state vector estimated during a previous iteration c_{k,k-1}, and the covariance matrix of the error at the initial time P_{k=1,0}, or resulting from a previous iteration P_{k,k-1}, determining the Kalman gain at time k (expression 60) and estimating the state vector c_{k,k} (expression 61).

By estimating c_{k,k}, C_{in blood} can be found out, the latter being the term of rank z_{deb1} of the vector c_{k,k}: this corresponds to step 135. From C_{in blood},

$${P}_{{CO}_{2}}^{in blood}$$

can be estimated (expression 67): step 136.

Step 140: Estimating the covariance matrix P_{k,k} of the estimation error (expression 62).

Step 150: Predicting the state vector c_{k,k+1} at time k+1 (expression 62′) and predicting of the covariance matrix of the error at the next time P_{k+1,k″}.

Step 160: incrementing time

Step 170: repeat steps 110 to 160 or exit the algorithm

Iterative steps 110 to 170 are repeated until the algorithm outputs. The output of the algorithm can be decided by the user, or correspond to a predetermined number of iterations.

The process described above has been implemented taking into account the following parameters:
length (along X axis) blood compartment: Δx^{blood}=Δx=5 cm;
width (along Y axis) blood compartment: Δy^{blood}=Δy=2 cm;
blood compartment height (along Z axis): Δz^{blood}=0.3 cm;
surface area of blood compartment in contact with skin: A^{blood}=10 cm²;
blood compartment volume: V^{blood}=3 cm³;
Henry's ratio of carbon dioxide in blood: H^{blood}=0.54
Diffusion coefficient of carbon dioxide in blood (water coefficient): D^{blood}=2.2 10⁻⁵ cm²·s⁻¹
Initial CO₂ concentration in blood: C^{in blood}=1.0990 μmol/cm3
Blood flow: Q^{blood}=1.83 10⁻³ ml/s (cm³s⁻¹) which corresponds to the following speed:
   Transverse velocity (along x):

$${u}_{x}^{blood} = 0.0031 cm . {s}^{- 1}$$

Axial velocity (along z):

$${u}_{z}^{blood} = 1.83 {10}^{- 4} cm . {s}^{- 1}$$

skin compartment length (along X axis): Δx^{skin}=Δx=5 cm;
skin compartment width (along Y axis): Δy^{skin}=Δy=2 cm;
skin compartment height (along Z axis): Δz^{skin}=16 10⁻⁴ cm;
skin surface in contact with the device: A^{skin}=10 cm²;
Henry's law coefficient of carbon dioxide in the skin: H^{skin}=1.6
Diffusion coefficient of carbon dioxide in the skin: D^{skin}=1 10⁻⁷ cm²·s⁻¹
measurement chamber temperature: T^{mes}=315.15 K;
measurement chamber length (along X axis): Δx^{mes}=Δx=5 cm;
measurement chamber width (along Y axis): Δy^{mes}=Δy=2 cm;
measurement chamber height (along Z axis): Δz^{mes}=0.25 cm;
Henry's law coefficient of carbon dioxide in the measurement chamber: H^{mes}=H^{air}=Diffusion coefficient of carbon dioxide in the measurement chamber: D^{mes}=D^{air}=0.18 cm²·s⁻¹
CO₂ concentration in incoming air, assuming filter: C^{air}=0
Convective air flow in collection chamber: Q^{air}=1.67 10⁻² ml/s
   Transverse speed (along x axis):

$${u}_{x}^{col} = 33 {10}^{- 3} cm . {s}^{- 1}$$

Axial velocity (along z axis):

$${u}_{z}^{col} = 1.67 {\text{.10}}^{- 3} cm . {s}^{- 1}$$

contact surface between measurement chamber and collection chamber: A^{mes}=A^{col}=10 cm²;
collection chamber temperature: T^{col}=315.15 K;
length of collection chamber (along X axis): Δx^{col}=Δx=5 cm;
width of collection chamber (along Y axis): Δy^{col}=Δy=2 cm;
height of collection chamber (along Z axis): Δz^{col}=0.25 cm;
Henry's ratio of carbon dioxide in the collection chamber: H^{col}=H^{air}=1
Diffusion coefficient of carbon dioxide in the collection chamber: D^{col}=D^{air}=0.18 cm²·s⁻¹
Transparency factor between skin and measuring cell: r_{skin|mes}=1;
Transparency factor between measurement cell and collection cell: r_{mes|col}=1
No time sampling: δt=1 s
Measurement noise variance: 10⁻⁶ mol/m³
Model noise variance: 10⁻⁸ mol/m³
Coefficient of the autoregressive model: φ=0

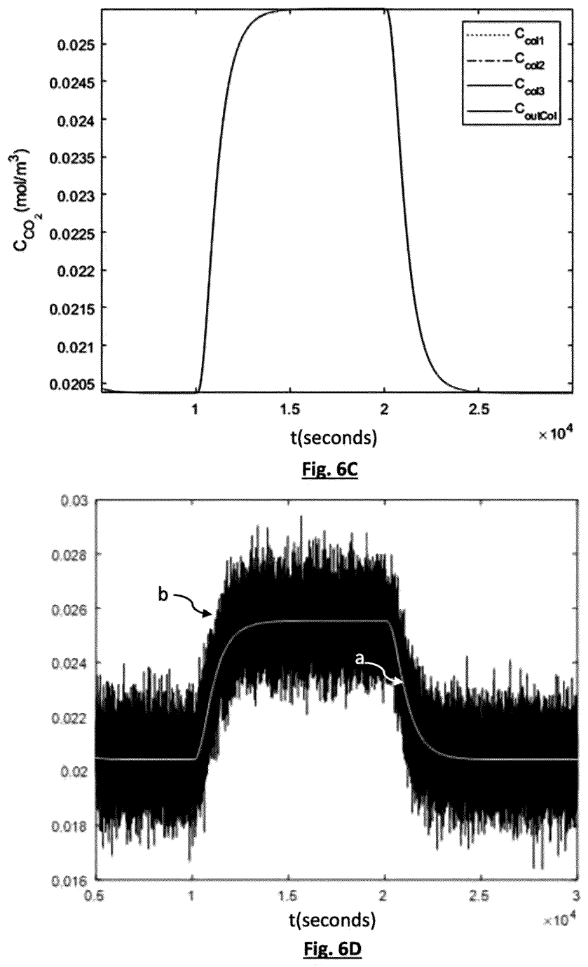

First, a direct model was used to estimate the concentration at each sampling point from a known concentration in blood. The known concentration in the blood follows a square-wave pattern. FIGS. 6A, 6B and 6C show the evolution of concentration as a function of time in the blood compartment, in the skin and in the collection chamber, respectively. FIG. 6D shows the evolution of concentration at the gas sensor, in the measurement chamber (curve a). Curve b in FIG. 6D shows the concentration at the gas sensor of curve a, to which Gaussian noise has been added to simulate noisy measurements. In each of FIGS. 6A to 6D, the x-axis corresponds to time and the y-axis corresponds to the estimated CO2 concentration.

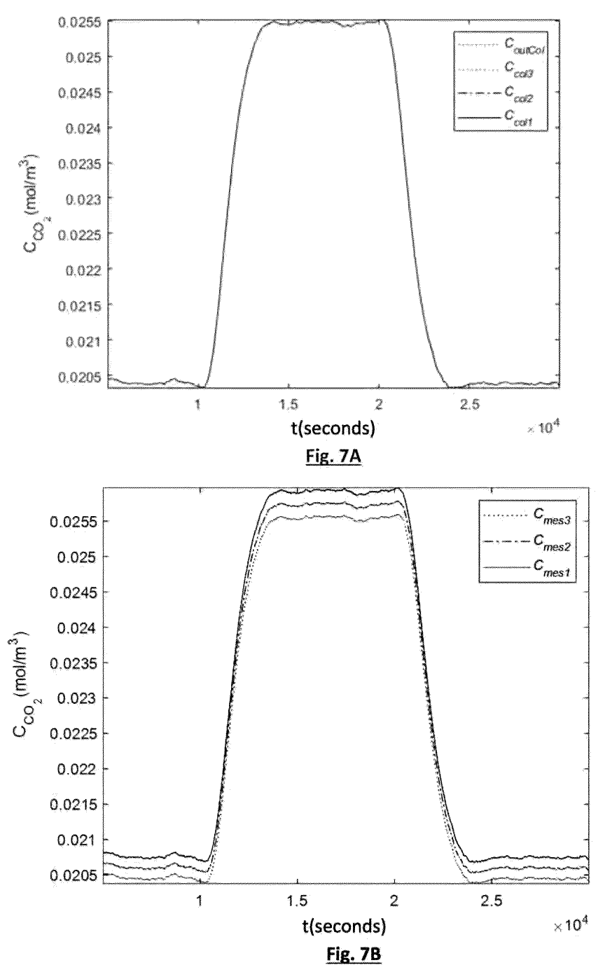

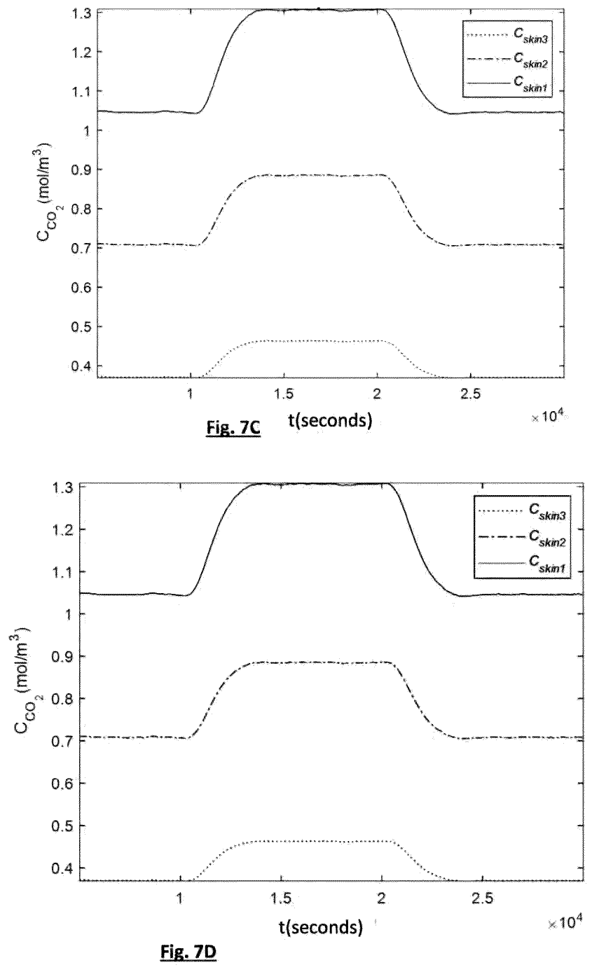

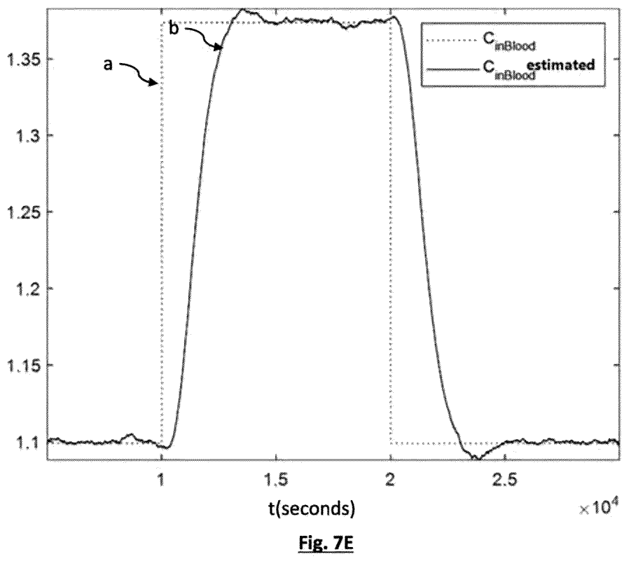

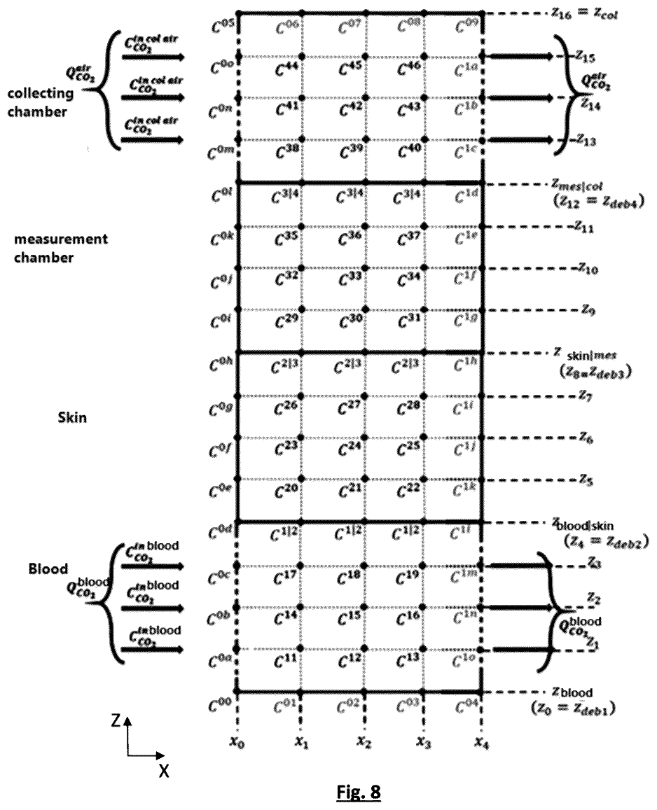

Using simulated noisy measurements (curve b in FIG. 6D), the recursive estimator described above was implemented. The state vector estimated at each measurement instant was used to obtain an estimate of the concentration at different sampling points. FIGS. 7A, 7B, 7C and 7D show the evolution of CO2 concentration in the collection chamber, measurement chamber, skin compartment and blood, respectively. FIG. 7E shows the actual blood concentration (curve a) and the estimated blood concentration.

In each of FIGS. 7A to 7E, the x-axis corresponds to the time and the y-axis corresponds to the estimated CO2 concentration.

### Second Model: Two-Dimensional Model

In the following, we describe a model in which evacuation in the collection cell is transverse (perpendicular to the z-axis), as shown in FIG. 8. The chosen geometry determines the calculation of the velocity vector field for the propagation of the carrier air in the collection cell, while the convection-diffusion equations define the propagation of the carbon dioxide. Air intake is via one or both side faces.

Blood propagation is modeled by circulation along an X axis, perpendicular to the Z axis.

Each of the compartments defined above is also discretized along the x axis, in the same way as along z, i.e. with 3 concentration values inside the compartments and two concentration values at the edges.

FIG. 8 shows the division into compartments. In this example, the position index in X varies from 0 to 4, as shown at the bottom of the figure. The position index along the z axis varies from 0 to 16 with the edges and from 1 to 15 for the internal points, as shown on the right of the figure. The index notation kₓ or k_{z} is used depending on the axis considered.

kₓ∈{0,1,2,3,4}

k_{z}∈{0,1,2,3,4,5,6,7,8,9,10,11,12,13,14,15,16}

For example, in the “Skin” compartment, along the X axis, the k, index varies from 0 to 4 for points including edges and from 1 to 3 for internal points; along the Z axis, the k_{z} index varies from 4 to 8, with interfaces, and from 5 to 7 for internal points.

In a homogeneous medium, equation (11) becomes:

$$\frac{\partial C ( x , z , t )}{\partial t} = - {u}_{x} \frac{\partial C ( x , z , t )}{\partial x} + D ( \frac{{\partial }^{2} C ( x , x , z , t )}{\partial {z}^{2}} + \frac{{\partial }^{2} C ( x , x , z , t )}{\partial {x}^{2}} ) \qquad ( 2 D - 1. )$$

The boundary conditions are:

At the bottom edge-level z₀, equation (13) becomes

$$\frac{\partial C ( {x}_{{k}_{x}} , {z}_{0} , t )}{\partial z} = 0 ; {k}_{x} ∈ \{ 0 , 1 , 2 , 3 , 4 \} \qquad ( 2 D - 2. )$$

At the top edge—level z^{col}=z₁₆, equation (13) becomes

$$\frac{\partial C ( {x}_{{k}_{x}} , {z}_{16} , t )}{\partial z} = 0 ; {k}_{x} ∈ \{ 0 , 1 , 2 , 3 , 4 \} \qquad ( 2 D - 3. )$$

Along the left side, at the coordinate x₀:
At the left lateral edge of the blood cell

$$\frac{\partial C ( {x}_{0} , {z}_{{k}_{z}} , t )}{\partial x} = 0 ; {k}_{z} ∈ \{ 0 \} \qquad ( 2 D - 4. )$$

$$C ( {x}_{0} , {z}_{{k}_{z}} , t ) = {C}_{in sang} ( t ) ; {k}_{z} ∈ \{ 1 , 2 , 3 \} \qquad ( 2 D - 5. )$$

Along the left side of the skin compartment and measurement chamber:

$$\frac{\partial C ( {x}_{0} , {z}_{{k}_{z}} , t )}{\partial x} = 0 ; {k}_{z} ∈ \{ 4 , … , 12 \} \qquad ( 2 D - 6. )$$

At the left side edge of the collection cell

$$\frac{\partial C ( {x}_{0} , {z}_{{k}_{z}} , t )}{\partial x} = 0 ; {k}_{z} ∈ \{ 16 \} \qquad ( 2 D - 7. )$$

$$C ( {x}_{0} , {z}_{{k}_{z}} , t ) = {C}_{in col air} ( t ) ; {k}_{z} ∈ \{ 13 , 14 , 15 \} \qquad ( 2 D - 8. )$$

Along the right side, according to the x₄ coordinate:

$$\frac{\partial C ( {x}_{4} , {z}_{{k}_{z}} , t )}{\partial x} = 0 ; {k}_{z} ∈ \{ 0 , 1 , … , 16 \} \qquad ( 2 D - 9. )$$

### Interface Conditions

Notation: the index i is used as a reference to the medium before the interface, the index j for the medium after the interface. The coordinate at the interface is indicated by the index i|j.

Continuity of flow, equation (18) becomes

$${r}_{transpa i \vert j} {D}_{i} \frac{\partial {C}_{i} ( x , {z}_{i \vert j} , t )}{\partial z} = D \frac{\partial {C}_{j} ( x , {z}_{i \vert j} , t )}{\partial z} \qquad ( 2 D - 10. )$$

Pressure continuity equation (21) becomes

$$\frac{{C}_{i} ( x , {z}_{i \vert j} , t )}{{H}_{i}} = \frac{{C}_{j} ( x , {z}_{i \vert j} , t )}{{H}_{j}} {=}^{def} {C}_{i \vert j} ( x , {z}_{i \vert j} , t ) \qquad ( 2 D - 11. )$$

### Expression of Air Speed.

The notations used are:
βₐᵢᵣ air density
Mₐᵢᵣ molar mass of air in the standard proportions of its various components
Cₐᵢᵣ molar concentration by volume of air in standard proportions of its various components
Pₐᵢᵣ air pressure
u^{air} air speed
γ^{air} air acceleration
Tₐᵢᵣ air temperature
Fⱼ mass force field reflecting the effect of gravity on the gas.
One can write:

ρₐᵢᵣ=Cₐᵢᵣ·Mₐᵢᵣ
According to the law of perfect gases:

$${P}_{air} = \frac{{ρ}_{air}}{{M}_{air}} R . {T}_{air}$$

The equation for conservation of momentum leads to the Navier Stokes equation. Since the velocity is low, we can assume that the gas is incompressible and that the density remains constant. We also neglect the effect of gravity on the gas and the viscosity of air. The simplified equation becomes:

ρₐᵢᵣ=Cₐᵢᵣ·Mₐᵢᵣ
where:

$${γ}_{air} = \frac{{du}_{air}}{dt} + {u}_{air} . \overline{\overline{grad}} ( {u}_{air} )$$

Where g̅r̅a̅d̅(uₐᵢᵣ) is the tensor:

$$\overline{\overline{grad}} ( {u}_{air} ) = [ \frac{\partial {u}_{air}}{\partial x} ]$$

The conservation of mass equation is written:

$$\frac{\partial {ρ}_{air}}{\partial t} + div ( {ρ}_{air} {u}_{air} ) = 0$$

### Steady State:

div(ρₐᵢᵣuₐᵢᵣ)=0

If ρₐᵢᵣ is assumed to be constant in the device chamber, a simplified equation can be obtained:

div(uₐᵢᵣ)=0

Knowledge of the air flow rate at the collection cell inlet determines the value of uₐᵢᵣ at the device inlet. By solving this equation, the velocity field inside the device can be calculated.

In the case of propagation in a single direction, e.g. X, the equation becomes:

$$\frac{{du}_{x , air}}{dx} = 0$$

The velocity field u_{x,air} is constant throughout the device and equal to the velocity vector at the device input.

Let's note (x₁, x₂) the spatial coordinates (x, z) and (u_{1,air}, u_{2,air}) the coordinates of the air velocity vector uₐᵢᵣ=(u_{x,air}, u_{z,air}):

div(uₐᵢᵣ)=0

Thus:

$${\sum }_{i = 1}^{2} \frac{\partial {u}_{i , air}}{\partial {x}_{i}} = 0$$

And:

$$\frac{\partial {u}_{j , air}}{\partial t} + {\sum }_{i = 1}^{2} {u}_{i , air} \frac{\partial {u}_{j , air}}{\partial {x}_{i}} = \frac{1}{{ρ}_{air}} [ - \frac{\partial {P}_{air}}{\partial {x}_{j}} + {F}_{j} ] j = 1 , 2$$

In the stationary state, assuming the effect of gravity on the gas to be negligible (Fⱼ=0), this equation becomes:

$${\sum }_{i = 1}^{2} {u}_{i , air} \frac{\partial {u}_{j , air}}{\partial {x}_{i}} = - \frac{1}{{ρ}_{air}} \frac{\partial {P}_{air}}{\partial {x}_{j}} j = 1 , 2$$

Navier Stokes equations for a perfect viscous incompressible fluid in 2D: If (x₁, x₂) represents the spatial coordinates (x, z) and (u_{1,air}, u_{2,air}) represents the coordinates of the velocity vector (u_{x,air}, u_{2,air}):

div(uₐᵢᵣ)=0

Thus:

$${\sum }_{i = 1}^{2} \frac{\partial {u}_{i , air}}{\partial {x}_{i}} = 0$$

And:

$$\frac{\partial {u}_{j , air}}{\partial t} + {\sum }_{i = 1}^{2} {u}_{i , air} \frac{\partial {u}_{j , air}}{\partial {x}_{i}} = \frac{1}{{ρ}_{air}} [ - \frac{\partial {P}_{air}}{\partial {x}_{j}} + {F}_{j} + v {\sum }_{i = 1}^{2} \frac{{\partial }^{2} {u}_{j , air}}{\partial {x}_{i}^{2}} ] j = 1 , 2$$

Where v is the kinetic viscosity of the fluid (unit: m²s⁻¹)

In the stationary state, assuming the effect of gravity on the gas to be negligible (Fⱼ=0), this equation becomes:

$${\sum }_{i = 1}^{2} {u}_{i , air} \frac{\partial {u}_{j , air}}{\partial {x}_{i}} = \frac{1}{{ρ}_{air}} [ - \frac{\partial {P}_{air}}{\partial {x}_{j}} + v {\sum }_{i = 1}^{2} \frac{{\partial }^{2} {u}_{j , air}}{\partial {x}_{i}^{2}} ] j = 1 , 2$$

In the case of the 2D model with transverse discharge, a simplified model of the velocity field is used, assuming that it is uniform throughout the chamber, and parallel to the x axis. Air enters from one side only, and equation (25) becomes:

$${u}_{x}^{col} = \frac{{Q}^{air}}{{A}_{x}^{in col}} \qquad ( 2 D - 12. )$$

Where Q^{air} is the air flow rate and

$${A}_{x}^{in col}$$

is the inlet area along the side wall of the collection cell 5,

$${A}_{x}^{out col}$$

is the outlet area along the side wall of the collection cell 5. It is further assumed that

$$\cdot {A}_{x}^{out col} = {A}_{x}^{in col}$$

The axial velocity is assumed to be zero.

$${u}_{z}^{col} = 0 \qquad ( 2 D - 13. )$$

### Expression of Blood Velocity.

$${A}_{x}^{in blood}$$

is the area entering the blood compartment on the left.

$${A}_{x}^{out blood}$$

is the area entering the blood compartment on the right. This models a cross-section of the blood vessels. It is assumed that

$${A}_{x}^{out blood} = {A}_{x}^{in blood} .$$

It is assumed that entry is via all sub-compartments of the blood compartment, i.e. for k, _531 z∈{1,2,3}.

$${u}_{x}^{blood} = \frac{{Q}^{blood}}{{A}_{x}^{out blood}} \qquad ( 2 D - 14. )$$

The axial velocity is assumed to be zero:

$${u}_{z}^{blood} = 0 \qquad ( 2 D - 15. )$$

Convection velocity in the measurement cell is assumed to be zero:

$${u}_{x}^{mes} = 0 \qquad ( 2 D - 16. )$$

$${u}_{z}^{mes} = 0 \qquad ( 2 D - 17. )$$

Convection velocity in the skin is assumed to be zero:

$${u}_{x}^{skin} = 0 \qquad ( 2 D - 18. )$$

$${u}_{z}^{skin} = 0 \qquad ( 2 D - 19. )$$

In summary, over the entire 2D model, the convection velocity field discretized over the sample points is written as:

$${u}_{x} ( x , {z}_{{k}_{z}} , t ) = \{ \begin{matrix}0 ; & {k}_{z} = 0 \\ {u}_{x}^{blood} ; & {k}_{z} ∈ \{ 1 , 2 , 3 \} \\ 0 ; & {k}_{z} ∈ \{ 4 , … , 12 \} \\ {u}_{x}^{col} ; & {k}_{z} ∈ \{ 13 , 14 , 15 \} \\ 0 ; & {k}_{z} = 16\end{matrix} \qquad ( 2 D - 20. )$$

$${u}_{z} ( x , {z}_{{k}_{z}} , t ) = \{ \begin{matrix}0 ; & {k}_{z} = 0 \\ 0 ; & {k}_{z} ∈ \{ 1 , 2 , 3 \} \\ 0 ; & {k}_{z} ∈ \{ 4 , … , 12 \} \\ 0 ; & {k}_{z} ∈ \{ 13 , 14 , 15 \} \\ 0 ; & {k}_{z} = 16\end{matrix} \qquad ( 2 D - 21. )$$

### 2D Model Discretization

For the discretization of the 2D continuous model, the approach remains the same as for the 1D model described above. From the continuous equations the discrete equations are derived, using the centered finite difference method. The problem is formulated in vector/matrix form in order to employ the Kalman filter for the estimation of the hidden states of the system, and for the estimation of the variable of interest C_{in blood}.

The spatial mesh is created along the X and Z axes, as shown in FIG. 8.

As in 1D, the distance δzᵢ between two successive points for each medium i along z is defined by:

$$δ {z}_{i} = \frac{Δ {z}_{i}}{{N}_{z , i} - 1}$$

Where N_{z,i} is the total number of sampling points along the z axis for medium i (N_{z,i}=5 in this case).

The position along z of the start of medium i in the column is noted z_{deb i}. It is defined by the

$${z}_{deb i} = \{ \begin{matrix}{z}_{0} & pour i = 1 \\ {z}_{0} + ( {\sum }_{j = 1}^{i - 1} Δ {z}_{j} ) & pour i = ∈ \{ 2 , 3 , 4 \}\end{matrix}$$

Where z₀ is the Z-axis dimension of the lower edge of the blood compartment and Δzⱼ is the height of the middle j.

The same approach is followed along the X axis:

The assumed equal width of each medium is: Δxᵢ=Δx

The number of discretization points are assumed to be the same for each medium along the X axis, including (Nₓ−2) internal points and two points on the edges, i.e. Nₓ points in total (Nₓ=5 in this case). The distance δx between two successive points for each medium i along x is:

$$δ x = \frac{Δ x}{{N}_{x} - 1}$$

Concentrations located in the vector c₀ are associated with spatial sampling points corresponding to different heights, then to different abscissas, and finally to different media.

The components of the vector c₀ are thus defined in the following way, first by varying the index kₓ corresponding to a scan along the X axis by adding the two edges, then by varying the index k_{z} corresponding to a scan along the Z axis by adding the edges and interfaces, then by varying the index i of the medium:

$${c}_{0} = [ \begin{matrix}C ( {x}_{0} + {k}_{x} . δ x , {z}_{deb 1} + {k}_{z} . δ {z}_{1} ) ; & {k}_{x} = 0 , … , {N}_{x} - 1 ; & {k}_{z} = 0 , … , ( {N}_{z , 1} - 1 ) \\ C ( {x}_{0} + {k}_{x} . δ x , {z}_{deb 2} + {k}_{z} . δ {z}_{2} ) ; & {k}_{x} = 0 , … , {N}_{x} - 1 ; & {k}_{z} = 1 , … , ( {N}_{z , 2} - 1 ) \\ C ( {x}_{0} + {k}_{x} . δ x , {z}_{deb 3} + {k}_{z} . δ {z}_{3} ) ; & {k}_{x} = 0 , … , {N}_{x} - 1 ; & {k}_{z} = 1 , … , ( {N}_{z , 3} - 1 ) \\ C ( {x}_{0} + {k}_{x} . δ x , {z}_{deb 4} + {k}_{z} . δ {z}_{4} ) ; & {k}_{x} = 0 , … , {N}_{x} - 1 ; & {k}_{z} = 0 , … , ( {N}_{z , 4} - 1 )\end{matrix} ]$$

$${c}_{0} = [ \begin{matrix}C ( {x}_{0} + ( {N}_{x} - 1 ) . δ x , {z}_{deb 1} + 0. δ {z}_{1} ) \\ … \\ C ( {x}_{0} + 0. δ x , {z}_{deb 1} + ( {N}_{z , 1} - 1 ) . δ {z}_{1} ) \\ … \\ C ( {x}_{0} + ( {N}_{x} - 1 ) . δ x , {z}_{deb 1} + ( {N}_{z , 1} - 1 ) . δ {z}_{1} ) \\ C ( {x}_{0} + 0. δ x , {z}_{deb 2} + 1. δ {z}_{2} ) \\ … \\ … \\ C ( {x}_{0} + ( {N}_{x} - 1 ) . δ x , {z}_{deb 2} + ( {N}_{z , 2} - 1 ) . δ {z}_{2} ) \\ C ( {x}_{0} + 0. δ x , {z}_{deb 3} + 1. δ {z}_{3} ) \\ … \\ … \\ C ( {x}_{0} + ( {N}_{x} - 1 ) . δ x , {z}_{deb 3} + ( {N}_{z , 3} - 1 ) . δ {z}_{3} ) \\ C ( {x}_{0} + 0. δ x , {z}_{deb 4} + 1. δ {z}_{4} ) \\ … \\ … \\ C ( {x}_{0} + ( {N}_{x} - 1 ) . δ x , {z}_{deb 4} + ( {N}_{z , 4} - 1 ) . δ {z}_{4} )\end{matrix} ]$$

N defines the total number of sample points taken into account for the spatial discretization of the transport model. These samples include points strictly inside the media, points at the upper and lower edges, points at the lateral edges and points corresponding to the three interfaces blood/skin, skin/measurement chamber, measurement chamber/collection chamber.

N=Nₓ·N_{z}

Where N_{z} is the total number of discrete samples in z,

$${N}_{z} = 1 + {\sum }_{i = 1}^{4} [ {N}_{z , i} - 1 ]$$

Nₛₜₐₜₑ groups points at the edges (lateral, upper and lower) and inside the strict z-media, excluding the 3×(Nₓ) points corresponding to the blood/skin, skin/measurement, measurement/collection interfaces.

In our case, we have: Nₓ=5; N_{z,1}=N_{z,2}=N_{z,3}=N_{z}4=5 so N_{z}=17; N=85 and Nₛₜₐₜₑ=70.

Once the vector c₀ has been defined, system resolution follows the same procedure as that described for 1D system resolution.

Given the spatial discretization, the 2D-1 diffusion convection equation in a homogeneous medium becomes:

$$\frac{C ( x , z , t + δ t ) - C ( x , z , t )}{δ t} = [ \frac{{u}_{x} δ x + 2 D}{2 {( δ x )}^{2}} ] C ( x - δ x , z , τ ) + [ \frac{- {u}_{x} δ x + 2 D}{2 {( δ x )}^{2}} ] C ( x + δ x , z , t ) + \frac{2 D}{δ {z}_{-} ( δ {z}_{+} + δ {z}_{-} )} C ( x , z - δ {z}_{-} , τ ) + \frac{- 2 D}{δ {z}_{+} δ {z}_{-}} C ( x , z , τ ) + \frac{2 D}{δ {z}_{+} ( δ {z}_{+} + δ {z}_{-} )} C ( x , z + δ {z}_{+} , τ )$$

C(x, z, t) is the concentration at point z which follows the concentration C(x, z−δz₋, t) defined at point z−δz₋ and which precedes the concentration C(x, z+δz₊, t) defined at point z+δz₊ if the (arbitrary) orientation of the z axis is followed. τ indicates the time reference frame.

For equal sampling steps δz₊=δz₋=δzᵢ, within the same homogeneous medium i the equation becomes:

$$\frac{C ( x , z , t + δ t ) - C ( x , z , t )}{δ t} = [ \frac{{u}_{x} δ x + 2 {D}_{i}}{2 {( δ x )}^{2}} ] C ( x - δ x , z , τ ) + [ \frac{- 2 {D}_{i}}{{( δ x )}^{2}} ] C ( x , z , τ ) + [ \frac{- {u}_{x} δ x + 2 {D}_{i}}{2 {( δ x )}^{2}} ] C ( x + δ x , z , τ ) + \frac{2 {D}_{i}}{2 {( δ {z}_{i} )}^{2}} C ( x , z - δ {z}_{i} , τ ) - \frac{2 {D}_{i}}{{( δ {z}_{i} )}^{2}} C ( x , z , τ ) + \frac{2 {D}_{i}}{2 {( δ {z}_{i} )}^{2}} C ( x , z + δ {z}_{+} , τ ) \qquad ( 2 D - 22 )$$

Thus:

$$\frac{C ( x , z , t + δ t ) - C ( x , z , t )}{δ t} = - 2 {D}_{i} [ \frac{1}{{( δ x )}^{2}} + \frac{1}{{( δ {z}_{i} )}^{2}} ] C ( x , z , τ ) + [ \frac{{u}_{x} δ x + 2 {D}_{i}}{2 {( δ x )}^{2}} ] C ( x - δ x , z , τ ) + [ \frac{- {u}_{x} δ x + 2 {D}_{i}}{2 {( δ x )}^{2}} ] C ( x + δ x , z , τ ) + \frac{2 {D}_{i}}{2 {( δ {z}_{i} )}^{2}} C ( x , z - δ {z}_{i} , τ ) - \frac{2 {D}_{i}}{{( δ {z}_{i} )}^{2}} C ( x , z + δ {z}_{+} , τ )$$

### Edge Discretization

We obtain the following equivalences for the edge equations:

$$\begin{matrix}\frac{C ( {x}_{0} + δ x , z , t ) - C ( {x}_{0} - δ x , z , t )}{δ x} = 0 ↔ C ( {x}_{0} - δ x , z , t ) = C ( {x}_{0} + δ x , z , t ) \\ \frac{C ( {x}_{4} + δ x , z , t ) - C ( {x}_{4} - δ x , z , t )}{δ x} = 0 ↔ C ( {x}_{4} - δ x , z , t ) = C ( {x}_{4} + δ x , z , t ) \\ \frac{C ( x , {z}_{0} + δ x , t ) - C ( x , {z}_{0} - δ z , t )}{δ z} = 0 ↔ C ( x , {z}_{0} - δ z , t ) = C ( x , {z}_{0} + δ z , t ) \\ \frac{C ( x , {z}_{16} + δ z , t ) - C ( x , {z}_{16} - δ z , t )}{δ z} = 0 ↔ C ( x , {z}_{16} - δ z , t ) = C ( x , {z}_{16} + δ z , t )\end{matrix}$$

By substituting in the discrete convection-diffusion equation:

Special case of the bottom left corner (x₀, z₀)

$$\frac{C ( {x}_{0} , {z}_{0} , t + δ t ) - C ( {x}_{0} , {z}_{0} , t )}{δ t} = [ \frac{- 2 {D}_{1}}{{( δ x )}^{2}} ] C ( {x}_{0} , {z}_{0} , τ ) + [ \frac{2 {D}_{i}}{{( δ x )}^{2}} ] C ( {x}_{0} - δ x , {z}_{0} , τ ) + [ \frac{- 2 {D}_{i}}{{( δ {z}_{i} )}^{2}} ] C ( {x}_{0} , {z}_{0} , τ ) + [ \frac{2 {D}_{1}}{{( δ {z}_{1} )}^{2}} ] C ( {x}_{0} , {z}_{0} + δ {z}_{1} , τ ) \qquad ( 2 D - 23 )$$

Special case of the bottom edge (xₖ_{₀}, z₀); kₓ∈{1,2,3,4}

$$\frac{C ( {x}_{{k}_{x}} , {z}_{0} , t + δ t ) - C ( {x}_{{k}_{x}} , {z}_{0} , t )}{δ t} = [ \frac{{u}_{x} δ x + 2 {D}_{1}}{2 {( δ x )}^{2}} ] C ( {x}_{{k}_{x}} - δ x , {z}_{0} , τ ) + [ \frac{- 2 {D}_{1}}{{( δ x )}^{2}} ] C ( {x}_{{k}_{x}} , {z}_{0} , τ ) + [ \frac{- {u}_{x} δ x + 2 {D}_{1}}{2 {( δ x )}^{2}} ] C ( {x}_{{k}_{x}} - δ x , {z}_{0} , τ ) + [ \frac{2 {D}_{1}}{{( δ {z}_{1} )}^{2}} ] C ( {x}_{{k}_{x}} , δ {z}_{1} , τ ) C ( {x}_{{k}_{x}} , {z}_{0} + δ {z}_{1} , τ ) ; {k}_{x} ∈ \{ 1 , 2 , 3 , 4 \} \qquad ( 2 D - 24 )$$

Special case of the bottom right corner (x₄, z₀)

$$\frac{C ( {x}_{4} , {z}_{0} , t + δ t ) - C ( {x}_{4} , {z}_{0} , t )}{δ t} = [ \frac{- 2 {D}_{1}}{{( δ x )}^{2}} ] C ( {x}_{4} , {z}_{0} , τ ) + [ \frac{2 {D}_{1}}{{( δ x )}^{2}} ] C ( {x}_{4} - δ x , {z}_{0} , τ ) + [ \frac{- 2 {D}_{1}}{{( δ {z}_{1} )}^{2}} ] C ( {x}_{4} , {z}_{0} , τ ) + [ \frac{2 {D}_{1}}{{( δ {z}_{1} )}^{2}} ] C ( {x}_{4} , {z}_{0} + δ {z}_{1} , τ ) \qquad ( 2 D - 25 )$$

Special case of the upper left corner (x₀, z₁₆)

$$( 2 D - 26 )$$

$$\begin{matrix}\frac{C ( {x}_{0} , {z}_{16} , t + δ t ) - C ( {x}_{0} , {z}_{16} , t )}{δ t} = [ \frac{- 2 {D}_{4}}{{( δ x )}^{2}} ] C ( {x}_{0} , {z}_{16} , τ ) \\ + [ \frac{2 {D}_{4}}{{( δ x )}^{2}} ] C ( {x}_{0} + δ x , {z}_{16} , τ ) \\ + [ \frac{- 2 {D}_{4}}{{( δ {z}_{4} )}^{2}} ] C ( {x}_{0} , {z}_{16} , τ ) \\ + [ \frac{2 {D}_{4}}{{( δ {z}_{4} )}^{2}} ] C ( {x}_{0} , {z}_{16} - δ {z}_{4} , τ )\end{matrix}$$

Special case of the top edge (xₖ_{ₓ}, z₁₆); kₓ∈{1,2,3,4}

$$( 2 D - 27 )$$

$$\begin{matrix}\frac{C ( {x}_{{k}_{x}} , {z}_{16} , t + δ t ) - C ( {x}_{{k}_{x}} , {z}_{16} , t )}{δ t} = [ \frac{{u}_{x} δ x + 2 {D}_{4}}{2 {( δ x )}^{2}} ] C ( {x}_{{k}_{x}} - δ x , {z}_{16} , τ ) \\ + [ \frac{- 2 {D}_{4}}{{( δ x )}^{2}} ] C ( {x}_{{k}_{x}} , {z}_{16} , τ ) \\ + [ \frac{- {u}_{x} δ x + 2 {D}_{4}}{2 {( δ x )}^{2}} ] C ( {x}_{{k}_{x}} + δ x , {z}_{16} , τ ) \\ + [ \frac{- 2 {D}_{4}}{{( δ {z}_{4} )}^{2}} ] C ( {x}_{{k}_{x}} , {z}_{16} , τ ) \\ + [ \frac{2 {D}_{4}}{{( δ {z}_{4} )}^{2}} ] C ( {x}_{{k}_{x}} , {z}_{16} - δ {z}_{4} , τ ) ; \\ {k}_{x} ∈ \{ 1 , 2 , 3 , 4 \}\end{matrix}$$

Special case of the top right-hand corner (x₄, z₁₆)

$$( 2 D - 28 )$$

$$\begin{matrix}\frac{C ( {x}_{4} , {z}_{16} , t + δ t ) - C ( {x}_{4} , {z}_{16} , t )}{δ t} = [ \frac{- 2 {D}_{4}}{{( δ x )}^{2}} ] C ( {x}_{4} , {z}_{16} , τ ) \\ + [ \frac{2 {D}_{4}}{{( δ x )}^{2}} ] C ( {x}_{4} - δ x , {z}_{16} , τ ) \\ + [ \frac{- 2 {D}_{4}}{{( δ {z}_{4} )}^{2}} ] C ( {x}_{4} , {z}_{16} , τ ) \\ + [ \frac{2 {D}_{4}}{{( δ {z}_{4} )}^{2}} ] C ( {x}_{4} , {z}_{16} - δ {z}_{4} , τ )\end{matrix}$$

Special case of the left edge (x₀, zₖ_{_{z}}); k_{z}∈{0,4, . . . , 12,16}

$$( 2 D - 29 )$$

$$\begin{matrix}\frac{C ( {x}_{0} , {z}_{{k}_{z}} , t + δ t ) - C ( {x}_{0} , {z}_{{k}_{z}} , t )}{δ t} = [ \frac{- 2 {D}_{i}}{{( δ x )}^{2}} ] C ( {x}_{0} , {z}_{{k}_{z}} , τ ) \\ + [ \frac{2 {D}_{i}}{{( δ x )}^{2}} ] C ( {x}_{0} + δ x , {z}_{{k}_{z}} , τ ) \\ + \frac{{D}_{i}}{{( δ {z}_{i} )}^{2}} C ( x , {z}_{{k}_{z}} - δ {z}_{i} , τ ) \\ + \frac{- 2 {D}_{i}}{{( δ {z}_{i} )}^{2}} C ( {x}_{0} , {z}_{{k}_{z}} , τ ) \\ + \frac{{D}_{i}}{{( δ {z}_{i} )}^{2}} C ( {x}_{0} , {z}_{{k}_{z}} + δ {z}_{i} , τ ) ; \\ {k}_{z} ∈ \{ 0 , 4 , … , 12 , 16 \}\end{matrix}$$

Special case of the right edge (x₄, zₖ_{_{z}}); k_{z}∈{0, . . . , 16}

$$\begin{matrix}\frac{( {x}_{4} , {z}_{{k}_{z}} , t + δ t ) - C ( {x}_{4} , {z}_{{k}_{z}} , t )}{δ t} = [ \frac{- 2 {D}_{i}}{{( δ x )}^{2}} ] C ( {x}_{4} , {z}_{{k}_{z}} , τ ) \\ + [ \frac{2 {D}_{i}}{{( δ x )}^{2}} ] C ( {x}_{4} - δ x , {z}_{{k}_{z}} , τ ) \\ + \frac{{D}_{i}}{{( δ {z}_{i} )}^{2}} C ( {x}_{4} , {z}_{{k}_{z}} - δ {z}_{i} , τ ) \\ + \frac{- 2 {D}_{i}}{{( δ {z}_{i} )}^{2}} C ( {x}_{4} , {z}_{{k}_{z}} , τ ) \\ + \frac{{D}_{i}}{{( δ {z}_{i} )}^{2}} C ( {x}_{4} , {z}_{{k}_{z}} + δ {z}_{i} , τ ) ; \\ {k}_{z} ∈ \{ 0 , … , 16 \}\end{matrix} \qquad ( 2 D - 30 )$$

### Discretization at Interfaces

Using the same principle as for the 1D model, the corresponding dummy concentration variables at the interface points z_{1|2} and z_{2|3} and z_{3|4} are introduced: C_{1|2}, C_{2|3} and C_{3|4}, see FIG. 11. C_{1|2} and C_{2|3} and C_{3|4} are eliminated from the system by substitution.

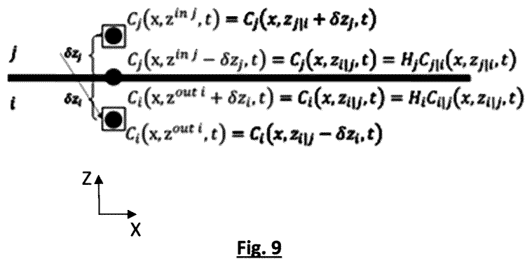

FIG. 9 illustrates how to discretize around an interface that separates two physical compartments.

The notations z^{out i} and z^{in j} correspond to k_{z}=4, 8 or 12 and “in” indicates an entry into a medium and “out” an exit from the medium.

Discretization of equations 2D-10 and 2D-11 yield to:

$$( 2 D - 31 )$$

$$\{ \begin{matrix}{r}_{transpa i \vert j} {D}_{i} \frac{{C}_{i} ( x , {z}_{i \vert j} , t ) - {C}_{i} ( x , {z}_{i \vert j} - δ {z}_{i} , t )}{δ {z}_{i}} = {D}_{j} \frac{{C}_{j} ( x , {z}_{i \vert j} + δ {z}_{+} , t ) - {C}_{j} ( x , {z}_{i \vert j} , t )}{δ {z}_{j}} \\ {C}_{i} ( x , {z}_{i \vert j} , t ) = {H}_{i} {C}_{i \vert j} ( x , {z}_{i \vert j} , t ) \\ {C}_{j} ( x , {z}_{j \vert i} , t ) = {H}_{j} {C}_{j \vert i} ( x , {z}_{j \vert i} , t ) \\ {C}_{i \vert j} ( x , {z}_{i \vert j} , t ) = {C}_{j \vert i} ( x , {z}_{j \vert i} , t )\end{matrix}$$

By replacing the concentration determined by Henry's law (Cᵢ(x, z_{i|j}, t), Cᵢ(x, z_{j|i}, t)) with the reference concentration at the interface C_{j|i}(x, z_{j|i}, t),

$$( 2 D - 32 )$$

$${r}_{transpa i \vert j} \frac{{D}_{i}}{δ {z}_{i}} {H}_{i} {C}_{i \vert j} ( x , {z}_{i \vert j} , t ) + \frac{{D}_{j}}{δ {z}_{j}} {H}_{j} {C}_{i \vert j} ( x , {z}_{i \vert j} , t ) = \frac{{D}_{j}}{δ {z}_{j}} {C}_{j} ( x , {z}_{i \vert j} + δ {z}_{j} , t ) + {r}_{transpa i \vert j} \frac{{D}_{i}}{δ {z}_{i}} {C}_{i} ( x , {z}_{i \vert j} - δ {z}_{i} , t )$$

The following notation is used:

$${ζ}_{i \vert j} = \frac{1}{{r}_{transpa i \vert j} {H}_{i} \frac{{D}_{i}}{δ {z}_{i}} + {H}_{j} \frac{{D}_{j}}{δ {z}_{j}}} \qquad ( 2 D - 33 )$$

Equation (2D-31) becomes

$$\begin{matrix}{C}_{j \vert i} ( x , {z}_{j \vert i} , t ) = {C}_{i , j} ( x , {z}_{i \vert j} , t ) \\ = {ζ}_{i \vert j} ( \frac{{D}_{j}}{δ {z}_{j}} {\underbrace{{C}_{j} ( x , {z}_{i \vert j} + δ {z}_{j} , t )}}_{{C}_{j} ( x , {z}^{in j} , t )} + {r}_{transpa i \vert j} \frac{{D}_{i}}{δ {z}_{i}} {\underbrace{{C}_{i} ( x , {z}_{i \vert j} - δ {z}_{i} , t )}}_{{C}_{i} ( x , {z}^{out i} , t )} ) )\end{matrix} \qquad ( 2 D - 34 )$$

In this equation, the two terms Cⱼ(x, z_{i|j}+δzⱼ, t) and Cᵢ(x, z_{i|j}−δzᵢ, t) can be recognized as the values of Cⱼ(x, z^{in j}, t) and Cᵢ(x, z^{out i}, t) respectively.

At the point just before the interface (Cᵢ(x, z^{out i}, t)=Cᵢ(x, z_{i|j}−δzᵢ, t), the concentration at the previous point Cᵢ(x, z^{out i}−δzᵢ, t) is internal to the medium, the concentration at the next point is placed on the interface is Cⱼ(x, z^{out i}+δzᵢ, t) which is equal to C_{i|j}(x, z_{i|j}, t)

$$\begin{matrix}{C}_{i} ( x , {z}^{out i} + δ {z}_{i} , t ) = {H}_{i} {C}_{i \vert j} ( x , {z}_{i \vert j} , t ) = {C}_{i} ( x , {z}_{i \vert j} , t ) \\ = {H}_{i} {ζ}_{i \vert j} ( \frac{{D}_{j}}{δ {z}_{j}} {C}_{j} ( x , {z}_{i \vert j} + δ {z}_{j} , t ) \\ + {r}_{transpa i \vert j} \frac{{D}_{i}}{δ {z}_{i}} {C}_{i} ( x , {z}_{i \vert j} - δ {z}_{i} , t ) )\end{matrix}$$

The concentration expression Cᵢ(x, z^{out i}+δzᵢ, t) thus determined is integrated into the discrete convection-diffusion equation (2D-22) in order to calculate the dynamics in the point z^{out i}

$$\frac{C ( x , {z}^{out i} , t + δt ) - C ( x , {z}^{out i} , t )}{δ t} = [ \frac{{u}_{x} δ x + 2 {D}_{i}}{2 {( δ x )}^{2}} ] C ( x - δ x , {z}^{out i} , τ ) + [ \frac{- 2 {D}_{i}}{{( δ x )}^{2}} ] C ( x , {z}^{out i} , τ ) + [ \frac{- {u}_{x} δ x + 2 {D}_{i}}{2 {( δ x )}^{2}} ] C ( x + δ x , {z}^{out i} , τ ) + \frac{2 {D}_{i}}{2 {( δ {z}_{i} )}^{2}} C ( x , {z}^{out i} - δ {z}_{i} , τ ) + \frac{- 2 {D}_{i}}{{( δ {z}_{i} )}^{2}} C ( x , {z}^{out i} , τ ) + \frac{2 {D}_{i}}{2 {( δ {z}_{i} )}^{2}} {H}_{i} {ζ}_{i \vert j} ( \frac{{D}_{j}}{δ {z}_{j}} {C}_{j} ( x , {z}_{i \vert j} + δ {z}_{j} , t ) + {r}_{transpa i \vert j} \frac{{D}_{i}}{δ {z}_{i}} {C}_{i} ( x , {z}_{i \vert j} - δ {z}_{i} , t ) ) \qquad ( 2 D - 35 )$$

or:

$$\frac{C ( x , {z}^{out i} , t + δt ) - C ( x , {z}^{out i} , t )}{δ t} = [ \frac{{u}_{x} δ x + 2 {D}_{i}}{2 {( δ x )}^{2}} ] C ( x - δ x , {z}^{out i} , τ ) + [ \frac{- 2 {D}_{i}}{{( δ x )}^{2}} ] C ( x , {z}^{out i} , τ ) + [ \frac{- {u}_{x} δ x + 2 {D}_{i}}{2 {( δ x )}^{2}} ] C ( x + δ x , {z}^{out i} , τ ) + [ \frac{2 {D}_{i}}{2 {( δ {z}_{i} )}^{2}} ] C ( x , {z}^{out i} - δ {z}_{i} , τ ) + [ \frac{- 2 {D}_{i}}{{( δ {z}_{i} )}^{2}} + {r}_{transpa i \vert j} {λ}_{i \vert j} \frac{{D}_{i}}{δ {z}_{i}} ] C ( x , {z}^{out i} , τ ) + [ {λ}_{i \vert j} \frac{{D}_{j}}{δ {z}_{j}} ] C ( x , {z}^{i n j} , t ) \qquad ( 2 D - 36 )$$

Where:

$${λ}_{i \vert j} = \frac{2 {D}_{i}}{2 {( {δz}_{i} )}^{2}} {H}_{l} {ζ}_{i \vert j} \qquad ( 2 D - 37 )$$

At the point just after the interface, Cⱼ(z^{in j}, t)=Cⱼ(z_{j|i}+δzⱼ, t), the concentration at the next point Cᵢ(x, z^{in j}+δzⱼ, t) is internal to the medium, the concentration at the previous point is located on the interface Cⱼ(x, z^{in j}−δzⱼ, t) is Cⱼ(x, z_{j|i}, t) which is equal to Cⱼ (z_{j|i}, t)

$${C}_{j} ( x , {z}^{i n j} - δ {z}_{j} , t ) = {C}_{j} ( x , {z}_{j \vert i} , t ) = {H}_{j} {C}_{j \vert i} ( x , {z}_{j \vert i} , t ) = {H}_{j} {ζ}_{i \vert j} ( \frac{{D}_{j}}{δ {z}_{j}} {C}_{j} ( x , {z}^{i n j} , t ) + {r}_{t r a nspa i \vert j} \frac{{D}_{i}}{δ {z}_{i}} {C}_{i} ( x , {z}^{out i} , t ) ) \qquad ( 2 D - 38 )$$

The concentration expression C(z^{in j}−δzⱼ, t) is integrated into the discrete convection-diffusion equation (2D-22) in order to calculate the dynamics in the point: z^{in j}

$$\frac{C ( x , {z}^{in j} , t + δt ) - C ( x , {z}^{in j} , t )}{δ t} = [ \frac{{u}_{x} δ x + 2 {D}_{i}}{2 {( δ x )}^{2}} ] C ( x - δ x , {z}^{i n j} , τ ) + [ \frac{- 2 {D}_{i}}{{( δ x )}^{2}} ] C ( x , {z}^{i n j} , τ ) + [ \frac{- {u}_{x} δ x + 2 {D}_{i}}{2 {( δ x )}^{2}} ] C ( x + δ x , {z}^{i n j} , τ ) + \frac{2 {D}_{j}}{2 {( δ {z}_{j} )}^{2}} {H}_{j} {ζ}_{i \vert j} ( \frac{{D}_{j}}{{δz}_{j}} {C}_{j} ( x , {z}^{i n j} , t ) + {r}_{t r a nspa i \vert j} \frac{{D}_{i}}{{δz}_{i}} {C}_{i} ( x , {z}^{out i} , t ) ) - \frac{2 {D}_{j}}{{( δ {z}_{j} )}^{2}} C ( x , {z}^{i n j} + τ ) + \frac{2 {D}_{j}}{2 {( δ {z}_{j} )}^{2}} C ( x , {z}^{in j} + δ {z}_{j} , τ ) \qquad ( 2 D - 39 )$$

### Or:

$$\frac{C ( x , {z}^{in j} , t + δt ) - C ( x , {z}^{in j} , t )}{δ t} = [ \frac{{u}_{x} δ x + 2 {D}_{i}}{2 {( δ x )}^{2}} ] C ( x - δ x , {z}^{i n j} , τ ) + \frac{- 2 {D}_{i}}{{( δ x )}^{2}} ] C ( x , {z}^{i n j} , τ ) + [ \frac{- {u}_{x} δ x + 2 {D}_{i}}{2 {( δ x )}^{2}} ] C ( x + δ x , {z}^{i n j} , τ ) + [ \frac{{λ}_{j \vert i} {D}_{j}}{δ {z}_{j}} - \frac{2 {D}_{j}}{{( δ {z}_{j} )}^{2}} ] {C}_{j} ( x , {z}^{i n j} , t ) + {r}_{transpa i \vert j} {λ}_{j \vert i} [ \frac{{D}_{i}}{δ {z}_{i}} ] {C}_{i} ( x , {z}^{out i} , t ) + [ \frac{2 {D}_{j}}{2 {( δ {z}_{j} )}^{2}} ] C ( x , {z}^{i n j} + δ {z}_{j} , τ ) \qquad ( 2 D - 40. )$$

Where:

$${λ}_{j \vert i} = \frac{2 {D}_{j}}{2 {( δ {z}_{j} )}^{2}} {H}_{j} {ζ}_{i \vert j}$$

The above equations can be written in matrix form by defining a spatial differential operator matrix F₀ which represents an operator with elements that are the bracketed terms of these equations:

c₀(x,z,t+δt)−c₀(x,z,t)=δt·F₀·c₀(x,z,τ)

This spatial differential operator matrix defines the temporal discretization:

If τ=t, an explicit Euler time integration scheme can be used:

c₀(x,z,t+δt)−c₀(x,z,t)=δt·F₀·c₀(x,z,t)

thus:

c₀(x,z,t+δt)=(I+δt·F₀)·c₀(x,z,t)

If τ=t+δt; an implicit Euler time integration scheme can be used:

c₀(x,z,t+δt)−c₀(x,z,t)=δt·F₀·c₀(x,z,t+δt)

thus:

(I−δt·F₀)·c₀(x,z,t+δt)=c₀(x,z,t)

The spatial differential operator matrices F₀ are thus expressed by isolating the terms related respectively to convection, diffusion, and conditions at interfaces and edges:

F₀=F_{0 comp}+F_{0 dif}+F_{0 int}
F_{0 conv} groups the terms of the equation related to convection,
F_{0 dif} groups the terms of the equation related to diffusion;
F_{0 int} groups together the terms of the equation related to interfaces and edges;

The discretization points are scanned in the x direction and then in the z direction to construct the matrices. The indices of the matrix elements are k_{F}.

If the position (x, z) has the index (k_{F}; k_{F}=∈{0, . . . , N−1}) in these matrices according to this scan in x then in z, the following positions will have the following indices:
(x−δx, z) index (k_{F}−1)
(x, z) index (k_{F})
(x+δx, z) index (k_{F}+1)
(x, zᵢ−δzᵢ) index (k_{F}−Nₓ)
(x, zᵢ+δzᵢ) index (k_{F}+Nₓ)

The edge positions are defined by the points (x, z) having the following indices (k_{F}) in these matrices:
bottom edge index k_{F}=(kₓ); kₓ==∈{0, . . . , (Nx−1)}
top edge index k_{F}=(kₓ+ (N_{z}−1)·Nₓ); kₓ=∈{0, . . . , (Nx−1)}
left edge index k_{F}=(0+k_{z}·Nₓ); k_{z}=∈{1, . . . , Nz−2}
right edge index k_{F}=((Nₓ−1)+k_{z}·Nₓ); k_{z}=∈{1, . . . , Nz−2}

The positions of the interfaces are defined by the points (x, z) having the following indices (k_{F}) in these matrices:
first interface k_{F}=(kₓ+(N_{z,1}−1)·Nₓ); kₓ=∈{0, . . . , (Nx−1)}
second interface k_{F}=(kₓ+(N_{z,1}−1+N_{z,2}−1)·Nₓ); kₓ=∈{0, . . . , (Nx−1)}
third interface k_{F}=(kₓ+(N_{z,1}−1+N_{z,2}−1+N_{z,3}−1)·Nₓ); kₓ=∈{0, . . . , (Nx−1)}

These matrices are hollow, so the matrix element at position k_{F} k_{F}′ can be noted fₖ_{_{F}}_{,k}_{_{F′}} F_{0 comp} is constructed as follows:

$${f}_{{k}_{F} , {k}_{F} - 1} = \frac{{u}_{x}}{2 δ x}$$

$${f}_{{k}_{F} , {k}_{F} + 1} = - \frac{{u}_{x}}{2 δ x}$$

the other terms of F_{0 comp} have a zero value.

F_{0 dif} is constructed as follows:

$${f}_{{k}_{F} , {k}_{F}} = - 2 {D}_{i} [ \frac{1}{{( δ x )}^{2}} + \frac{1}{{( δ {z}_{i} )}^{2}} ]$$

$${f}_{{k}_{F} , {k}_{F} - 1} = \frac{{D}_{l}}{{( δ x )}^{2}}$$

$${f}_{{k}_{F} , {k}_{F} + 1} = - \frac{{D}_{l}}{{( δ x )}^{2}}$$

$${f}_{{k}_{F} , {k}_{F} - {N}_{x}} = \frac{{D}_{l}}{{( δ {z}_{i} )}^{2}}$$

$${f}_{{k}_{F} , {k}_{F} + {N}_{x}} = - \frac{{D}_{l}}{{( δ {z}_{i} )}^{2}}$$

the other terms of F_{0 dif} have a zero value.

F_{0 int} is constructed as follows:

For samples below the interface (k_{F}=k_{F}_{_{interface}}−Nₓ)

$${k}_{F} = ( {k}_{x} + ( {N}_{z , 1} - 2 ) , {N}_{x} ) ; {k}_{x} = ∈ \{ 0 , … , ( Nx - 1 ) \}$$

$${k}_{F} = ( {k}_{x} + ( {N}_{z , 1} - 1 + {N}_{z , 2} - 2 ) , {N}_{x} ) ; {k}_{x} = ∈ \{ 0 , … , ( Nx - 1 ) \}$$

$${k}_{F} = ( {k}_{x} + ( {N}_{z , 1} - 1 + {N}_{z , 2} - 1 + {N}_{z , 3} - 2 ) , {N}_{x} ) ; {k}_{x} = ∈ \{ 0 , … , ( Nx - 1 ) \}$$

$${f}_{{k}_{F} , {k}_{F} - 1} = \frac{{u}_{x} δ x + 2 {D}_{i}}{2 {( δ x )}^{2}}$$

$${f}_{{k}_{F} , {k}_{F}} = \frac{- 2 {D}_{i}}{{( δ x )}^{2}} - \frac{2 {D}_{i}}{{( δ {z}_{i} )}^{2}} + {transpa}_{i \vert j} {λ}_{i \vert j} \frac{{D}_{i}}{δ {z}_{i}}$$

$${f}_{{k}_{F} , {k}_{F} + 1} = \frac{- {u}_{x} δ x + 2 {D}_{i}}{2 {( δ x )}^{2}}$$

$${f}_{{k}_{F} , {k}_{F} - {N}_{x}} = \frac{2 {D}_{i}}{2 {( {δz}_{i} )}^{2}}$$

$${f}_{{k}_{F} , {k}_{F} + 2 {N}_{x}} = {λ}_{i \vert j} \frac{{D}_{j}}{{δz}_{j}}$$

$${f}_{{k}_{F - {N}_{x}} , {k}_{F} + {N}_{x}} = {λ}_{i \vert j} \frac{{D}_{j}}{{δz}_{j}}$$

For samples above the interface: (k_{F}=k_{F}_{_{interface}}+Nₓ):

$${k}_{F} = ( {k}_{x} + ( {N}_{z , 1} ) , {N}_{x} ) ; {k}_{x} = ∈ \{ 0 , … , ( Nx - 1 ) \}$$

$${k}_{F} = ( {k}_{x} + ( {N}_{z , 1} - 1 + {N}_{z , 2} ) , {N}_{x} ) ; {k}_{x} = ∈ \{ 0 , … , ( Nx - 1 ) \}$$

$${k}_{F} = ( {k}_{x} + ( {N}_{z , 1} - 1 + {N}_{z , 2} - 1 + {N}_{z , 3} ) , {N}_{x} ) ; {k}_{x} = ∈ \{ 0 , … , ( Nx - 1 ) \}$$

$${f}_{{k}_{F} , {k}_{F} - 1} = \frac{{u}_{x} δ x + 2 {D}_{i}}{2 {( δ x )}^{2}}$$

$${f}_{{k}_{F} , {k}_{F}} = \frac{- 2 {D}_{i}}{{( δ x )}^{2}} - \frac{2 {D}_{j}}{{( δ {z}_{j} )}^{2}} + \frac{{λ}_{j \vert i} {D}_{j}}{δ {z}_{j}}$$

$${f}_{{k}_{F} , {k}_{F} + 1} = \frac{- {u}_{x} δ x + 2 {D}_{i}}{2 {( δ x )}^{2}}$$

$${f}_{{k}_{F} , {k}_{F} - 2 {N}_{x}} = {r}_{transpa i \vert j} {λ}_{j \vert i} \frac{{D}_{i}}{δ {z}_{i}}$$

$${f}_{{k}_{F} , {k}_{F} + {N}_{x}} = \frac{2 {D}_{j}}{2 {( δ {z}_{j} )}^{2}}$$

Where ζ_{i|j} and λ_{i|j} and λ_{j|i} have been defined above (equations 2D-33 and 2D-37 and 2D-41). For edges:

$$\{ \begin{matrix}{f}_{0 , 0} = \frac{- 2 {D}_{1}}{{( δ x )}^{2}} - \frac{- 2 {D}_{1}}{{( δ {z}_{1} )}^{2}} \\ {f}_{0 , 1} = \frac{2 {D}_{1}}{{( δ x )}^{2}} \\ {f}_{0 , {N}_{x}} = \frac{2 {D}_{1}}{{( δ {z}_{1} )}^{2}}\end{matrix}$$

$$\{ \begin{matrix}{f}_{4 , 4} = \frac{- 2 {D}_{1}}{{( δ x )}^{2}} - \frac{- 2 {D}_{1}}{{( δ {z}_{1} )}^{2}} \\ {f}_{4 , 3} = \frac{2 {D}_{1}}{{( δ x )}^{2}} \\ {f}_{4 , 4 + {N}_{x}} = \frac{2 {D}_{1}}{{( δ {z}_{1} )}^{2}}\end{matrix}$$

$$\{ \begin{matrix}{f}_{( {N}_{z} - 1 ) \cdot {N}_{x} , ( {N}_{z} - 1 ) \cdot {N}_{x}} = \frac{- 2 {D}_{4}}{{( δ x )}^{2}} - \frac{- 2 {D}_{4}}{{( δ {z}_{4} )}^{2}} \\ {f}_{( {N}_{z} - 1 ) \cdot {N}_{x} , ( {N}_{z} - 1 ) \cdot {N}_{x} + 1} = \frac{2 {D}_{4}}{{( δ x )}^{2}} \\ {f}_{( {N}_{z} - 1 ) \cdot {N}_{x} , ( {N}_{z} - 2 ) \cdot {N}_{x}} = \frac{2 {D}_{4}}{{( δ {z}_{4} )}^{2}}\end{matrix}$$

$$\{ \begin{matrix}{f}_{4 + ( {N}_{z} - 1 ) \cdot {N}_{x} , 4 + ( {N}_{z} - 1 ) \cdot {N}_{x}} = \frac{- 2 {D}_{4}}{{( δ x )}^{2}} - \frac{- 2 {D}_{4}}{{( δ {z}_{4} )}^{2}} \\ {f}_{4 + ( {N}_{z} - 1 ) \cdot {N}_{x} , 3 + ( {N}_{z} - 1 ) \cdot {N}_{x}} = \frac{2 {D}_{4}}{{( δ x )}^{2}} \\ {f}_{4 + ( {N}_{z} - 1 ) \cdot {N}_{x} , 4 + ( {N}_{z} - 2 ) \cdot {N}_{x}} = \frac{2 {D}_{4}}{{( δ {z}_{4} )}^{2}}\end{matrix}$$

$${k}_{F} = ( {k}_{x} ) ; {k}_{x} = ∈ \{ 1 , … , ( Nx - 2 ) \}$$

$$\{ \begin{matrix}{f}_{{k}_{F} , {k}_{F} - 1} = \frac{{u}_{x} δ x + 2 {D}_{1}}{2 {( δ x )}^{2}} \\ {f}_{{k}_{F} , {k}_{F}} = \frac{- 2 {D}_{1}}{{( δ x )}^{2}} + \frac{- 2 {D}_{1}}{{( δ {z}_{1} )}^{2}} \\ {f}_{{k}_{F} , {k}_{F} + 1} = \frac{- {u}_{x} δ x + 2 {D}_{1}}{2 {( δ x )}^{2}} \\ {f}_{{k}_{F} , {k}_{F} + {N}_{x}} = \frac{2 {D}_{1}}{2 {( δ {z}_{1} )}^{2}}\end{matrix}$$

$${k}_{F} = ( {k}_{x} + ( {N}_{z} - 1 ) \cdot {N}_{x} ) ; {k}_{x} = ∈ \{ 1 , … , ( Nx - 2 ) \}$$

$$\{ \begin{matrix}{f}_{{k}_{F} , {k}_{F} - 1} = \frac{{u}_{x} δ x + 2 {D}_{4}}{2 {( δ x )}^{2}} \\ {f}_{{k}_{F} , {k}_{F}} = \frac{- 2 {D}_{4}}{{( δ x )}^{2}} + \frac{- 2 {D}_{4}}{{( δ {z}_{1} )}^{2}} \\ {f}_{{k}_{F} , {k}_{F} + 1} = \frac{- {u}_{x} δ x + 2 {D}_{4}}{2 {( δ x )}^{2}} \\ {f}_{{k}_{F} , {k}_{F} - {N}_{x}} = \frac{2 {D}_{4}}{2 {( δ {z}_{4} )}^{2}}\end{matrix}$$

$${k}_{F} = ( 0 + {k}_{z} \cdot {N}_{x} ) ; {k}_{z} = ∈ \{ 1 , … , Nx - 2 \}$$

The other terms of F₀ int have a zero value.

### Restricted Direct Problem

To describe the direct transport model, only the terms located strictly inside the z-media may be used, ignoring the 3x(Nₓ) points corresponding to the blood/skin, skin/measurement chamber, measurement chamber/collection chamber interfaces and the points at the edges (lateral, upper and lower). From the description above, it has been shown how to eliminate, from the equations, the points located exactly at the interfaces by points before or after the interface. The points in the F_{0 int} matrix corresponding to the interfaces are eliminated from the matrices.

A new F matrix of dimension (Nₑₜₐₜ) is defined, in which the interface points are removed. Let's take the index points k_{F} defined above.

F=F_{conv}+F_{dif}+Fᵢₙₜ

A control matrix G is defined, that comprises the known input values.

G is a two-column matrix of size (Nₑₜₐₜ, 2) of the form (case of transverse evacuation):

$$G = \{ \begin{matrix}{g}_{{k}_{G} , 1} ; & {k}_{G} = ( 0 + {k}_{z} \cdot {N}_{x} ) ; {k}_{z} = ∈ \{ 1 , 2 , 3 \} \\ {g}_{{k}_{G} , 2} ; & \begin{matrix}{k}_{G} = ( 0 + ( {N}_{z , 1} + {N}_{z , 2} + {N}_{z , 3} - 6 + {k}_{z} ) \cdot {N}_{x} ) ; \\ {k}_{z} = ∈ \{ 1 , 2 , 3 \}\end{matrix} \\ 0 & for all other terms\end{matrix}$$

Where

$${ℊ}_{{k}_{G} , 1} = \frac{{u}_{x}^{b l o o d}}{δ {x}^{b l o o d}} and . {ℊ}_{{k}_{G} , 2} = \frac{{u}_{x}^{c o l}}{δ x}$$

q is the vector containing the exogenous inputs to the system. These are the initial concentration introduced into the system in the liquid phase C^{in Blood} on the one hand, and the concentration of CO₂ in the ambient air C^{in Col Air} on the other.

$$q = [ \begin{matrix}{C}^{in Blood} \\ {C}^{in Col Air}\end{matrix} ]$$

Concentration data are generated using a signal model based on the recursivity relationship:

Acₖ_{ₜ}₊₁=cₖ_{ₜ}+Gqₖ_{ₜ}₊₁

Where A is the transition matrix obtained from the implicit time discretization and c—the state vector of dimension Nₑₜₐₜ

A=I−F·δt

In the case of transverse counter-current discharge, the air inlet is from the opposite side, and the matrix G is expressed as follows:

$$G = \{ \begin{matrix}{ℊ}_{{k}_{G} , 1} ; & {k}_{G} = ( 0 + {k}_{z} \cdot {N}_{x} ) ; {k}_{z} \text{=} ∈ \{ 1 , 2 , 3 \} \\ {ℊ}_{{k}_{G} , 2} ; & {k}_{G} = ( 0 + ( {N}_{z , 1} + {N}_{z , 2} + {N}_{z , 3} - 6 + {k}_{z} ) \cdot {N}_{x} + ( {N}_{x} - 1 ) ) ; {k}_{z} \text{=} ∈ \{ 1 , 2 , 3 \} \\ 0 & for all other terms\end{matrix}$$

Where

$${ℊ}_{{k}_{G} , 1} = \frac{{u}_{x}^{b l o o d}}{δ {x}^{b l o o d}} and . {ℊ}_{{k}_{G} , 2} = \frac{- {u}_{x}^{c o l}}{δ x}$$

The Kalman algorithm enables to estimate a variable of interest, in this case C^{in blood}, in a linear fashion, seeking to minimize the mean-square error between the actual value and the estimated value. It is based on the formulation of a state-space model associating a state evolution equation, and an observation equation expressing the link between measurements and states.

c[kₜ+1]=Fc[kₜ]+Gq[kₜ+1]+w[kₜ+1] (2D-42)

y(kₜ)=Hc(kₜ)+v(kₜ) (2D-43)

kₜ represents the time discretization index.

The vector w describes the model noise on the state evolution equation, with variance-covariance matrix Q.

Through the observation matrix H, the observable of the state vector c is defined. In this specific case, we have only one observable for the state vector, all other concentration variables being hidden, so the matrix can be reduced to a transpose vector.

H=h^{t}

The vector v describes the observation noise with variance-covariance matrix R.

The state vector can be augmented with the desired variable C_{in blood} in the same way as in 1D.

The variable to be estimated C_{in blood}, is located in the vector \tilde{c}ₖ at position x₀, z₀. It is linked to the following state variable C_{x1,z1} by the terms of the transport equation. It is assumed that the signal can be modeled by a first-order autoregressive model defined by the following recurrence relation:

$$\frac{\partial }{\partial t} {C}^{in blood} = {φ C}^{in blood} + {w}^{in blood} \qquad ( 2 D - 44 )$$

in discrete form:

(1−φ)C_{in blood}[kₜ+1]=δt C_{in blood}[kₜ]+w[kₜ+1] (2D-45)

Where φ is a model parameter. φ should be interpreted as a signal regularity parameter.
If φ=0, the time derivative of the input signal is assumed to be zero. There is no variation.
If φ>0 the derivative of the input signal is assumed to be positive for a positive concentration C_{in blood}. This favors signal increase, but will penalize signal decay.

An augmented system is defined, by associating the physical model of the system described above with this signal model.

$$\{ \begin{matrix}\frac{\partial }{\partial t} c = F c + G q + w \\ \frac{\partial }{\partial t} {C}^{in blood} = {φ C}^{in blood} + {w}^{in blood}\end{matrix} \qquad ( 2 D - 46 )$$

The equation of state of the augmented system can be written as:

$$\frac{\partial }{\partial t} \tilde{c} = \tilde{F} \tilde{c} + \tilde{G} \tilde{q} + \tilde{w} \qquad ( 2 D - 47 )$$

Implicit discretization yields the following equation:

Ã\tilde{c}ₖ_{ₜ}₊₁=\tilde{c}ₖ_{ₜ}+\tilde{G}qₖ_{ₜ}₊₁+\tilde{w}ₖ_{ₜ}₊₁

With:

$$\tilde{A} = [ \begin{matrix}φ & 0 \\ {ℊ}_{0 , 1} & \\ 0 & \\ 0 & [ A ] \\ 0 & \\ 0 & \end{matrix} ]$$

$$\tilde{c} = [ \begin{matrix}{C}^{in blood} \\ {C}_{{k}_{F}}\end{matrix} ] ; {k}_{F} = 1 : {N}_{etat}$$

\tilde{w}[k]~ [symbol] (0, \tilde{Q}) is the noise that incorporates the model errors of dimension N′×1. Where [symbol] (0, \tilde{Q}), is a vector normal distribution with mean zero for each component and variance-covariance matrix

$$\tilde{Q} . {\tilde{σ}}_{w}^{2}$$

is the variance of the model noise describing the model errors common to each component of the noise vector \tilde{w} for each time kₜ. The variance-covariance matrix \tilde{Q} is

$$\tilde{Q} = {\tilde{σ}}_{w}^{2} \cdot I$$

\tilde{G} the control matrix is now limited to C^{in col air} (deletion of the first column).

$$\tilde{G} = [ \begin{matrix}0 \\ ⋯ \\ 0 \\ {ℊ}_{{k}_{G} , 2} \\ 0 \\ ⋯ \\ 0\end{matrix} ]$$

$${k}_{G} = ( 0 + ( {N}_{z , 1} + {N}_{z , 2} + {N}_{z , 3} - 6 + {k}_{z} ) \cdot {N}_{x} ) ; {k}_{z} \text{=} ∈ \{ 1 , 2 , 3 \}$$

$${ℊ}_{{k}_{G} , 2} = \frac{{u}_{x}^{col}}{\partial x}$$

As mentioned above, in the case of transverse counter-current evacuation:

$${k}_{G} = ( 0 + ( {N}_{z , 1} + {N}_{z , 2} + {N}_{z , 3} - 6 + {k}_{z} ) \cdot {N}_{x} + ( {N}_{x} - 1 ) ; {k}_{z} \text{=} ∈ \{ 1 , 2 , 3 \}$$

$${ℊ}_{{k}_{G} , 2} = \frac{- {u}_{x}^{col}}{\partial x}$$

### Kalman Filtering Algorithm.

\tilde{c}ₖ_{ₜ}_{,k}_{ₜ}₋₁ is the augmented state vector at time kₜ predicted at the previous time kₜ−1
\tilde{c}ₖ_{ₜ}_{,k}_{ₜ} is the augmented state vector at time kₜ calculated at time kₜ
\tilde{c}ₖ_{ₜ}_{+1,k}_{ₜ} is the augmented state vector at time kₜ+1 predicted at time kₜ
Pₖ_{ₜ}_{,k}_{ₜ}₋₁ is the covariance matrix of the error at time kₜ predicted at the previous time kₜ−1
Pₖ_{ₜ}_{,k}_{ₜ} is the covariance matrix of the error at time kₜ calculated at time kₜ
Pₖ_{ₜ}_{+1,k}_{ₜ} is the covariance matrix of the error at time kₜ+1 predicted at time kₜ
Kₖ_{ₜ} is the Kalman gain calculated at kₜ
yₖ_{ₜ} is the new measurement observed at kₜ
Ã is the transition matrix applied to the state \tilde{c}ₖ_{ₜ}₊₁
\tilde{G} is the control matrix applied to the input \tilde{q} of the augmented system.
\tilde{H} is the augmented observation matrix
\tilde{Q} is the noise covariance matrix of the augmented restricted system.
R is the covariance matrix of the observation noise

The problem to be solved is to obtain recurrent relations for estimating the state vector \tilde{c}ₖ_{ₜ}_{,k}_{ₜ} as a function of the previous state vector \tilde{c}ₖ_{ₜ}_{-1,k}_{ₜ}₋₁ and the new observation yₖ_{ₜ}. Two steps are required to solve this problem:
#1 The step of correcting and updating the state vector and covariance matrix from the observation equations and the state vector and covariance matrix estimated in the previous prediction step.
#2 The step of predicting the state vector and covariance matrix at the next instant from the state vector and covariance matrix at the previous instant.

These two steps are preceded by an initialization:
#0 The state of the system c_{0,0} at time kₜ=0 and the error covariance matrix P_{0,0} are assumed to be known.

The state vector at time kₜ=0 is initialized by the null vector: \tilde{c}_{0,0}=0 (of dimensions Nₑₜₐₜ×1) and the matrix designating the error covariance by the identity matrix (of dimensions Nₑₜₐₜ×Nₑₜₐₜ).

The algorithm is stopped when no new measurements are taken.

The computational loop of the Kalman filtering algorithm is now presented:

### Correction Phase

The state vector \tilde{c}ₖ_{ₜ}_{,k}_{ₜ}₋₁ at time kₜ predicted at time kₜ−1 is known, as well as the covariance matrix of the error Pₖ_{ₜ}_{,k}_{ₜ}₋₁ at time kₜ predicted at time kₜ−1. yₖ_{ₜ} is observed.

The estimation of \tilde{c}ₖ_{ₜ}_{,k}_{ₜ}Pₖ_{ₜ}_{,k}_{ₜ} is sought.

The Kalman gain Kₖ_{ₜ} is determined with the following formula where Pₖ_{ₜ}_{,k}_{ₜ}₋₁ is calculated in equation 2D-44 or 2D-45:

Kₖ_{ₜ}=Pₖ_{ₜ}_{,k}_{ₜ}₋₁\tilde{H}^{T}(\tilde{H}Pₖ_{ₜ}_{,k}_{ₜ}₋₁\tilde{H}^{T}+R)⁻¹ (2D-48)

The Kalman gain expresses the weights (confidence) given to the new noisy measurement yₖ_{ₜ} or to the state estimate cₖ_{ₜ}_{,k}_{ₜ}₋₁, which is also subject to the various external disturbances. The estimate of the current state (a posteriori state estimator) of the system is made by taking a linear combination between the estimate made at the previous instant kₜ−1 (a priori state estimator) and the new recorded data. The Kalman gain is used to correct the estimate made at time kₜ−1 for the current time kₜ according to the new recorded measurement yₖ_{ₜ}.

Knowing the Kalman gain expression Kₖ_{ₜ}, the expression for estimating the state vector \tilde{c}ₖ_{ₜ}_{,k}_{ₜ} at time kₜ can be expressed as a function of the difference between the current measurement yₖ_{ₜ} and the estimated measurement \tilde{H}\tilde{c}ₖ_{ₜ}_{,k}_{ₜ}₋₁ from the previous state estimate. This difference defines the prediction error (yₖ_{ₜ}−\tilde{H}\tilde{c}ₖ_{ₜ}_{,k}_{ₜ}₋₁), which evaluates the amount of new information brought in by the current measurement.

\tilde{c}ₖ_{ₜ}_{,k}_{ₜ}=\tilde{c}ₖ_{ₜ}_{,k}_{ₜ}₋₁+Kₖ_{ₜ}(yₖ_{ₜ}−\tilde{H}\tilde{c}ₖ_{ₜ}_{,k}_{ₜ}₋₁) (2D-49)

The covariance matrix of the estimation error at time kₜ is estimated from the covariance matrix Pₖ_{ₜ}_{,k}_{ₜ}₋₁ estimated at time kₜ−1 using the following expression:

$${P}_{{k}_{t} , {k}_{t}} = ( I - {K}_{{k}_{t}} \tilde{H} ) {{P}_{{k}_{t} , {k}_{t} - 1} ( I - {K}_{{k}_{t}} \tilde{H} )}^{T} + {K}_{{k}_{t}} R {K}_{{k}_{t}}^{T} \qquad ( 2 D - 50 )$$

The estimation of \tilde{c}ₖ_{ₜ}_{,k}_{ₜ} allows to know C_{in blood}, the latter being the rank 1 term of the vector. \tilde{c}ₖ_{ₜ}_{,k}_{ₜ}

From C_{in blood},

$${P}_{{CO}_{2}}^{in blood}$$

can be estimated

$${P}_{{CO}_{2}}^{in blood} = \frac{{C}_{{CO}_{2}}^{in blood}}{{β}^{blood}} \qquad ( 2 D - 51 )$$

where:
β^{blood} is the Ostwald solubility coefficient of carbon dioxide in blood.

### Prediction Phase

The state vector \tilde{c}ₖ_{ₜ}_{,k}_{ₜ} and the covariance matrix of the estimation error Pₖ_{ₜ}_{,k}_{ₜ} at time kₜ are known.

The new state of the system is \tilde{c}ₖ_{ₜ}_{+1,k}_{ₜ}. The integration scheme corresponding to the implicit method is considered. The prediction equation is:

$$\tilde{A} {\tilde{c}}_{{k}_{t} + 1 , {k}_{t}} = {\tilde{c}}_{{k}_{t} , {k}_{t}} + \tilde{G} {C}_{{k}_{t} + 1}^{in col air} \qquad ( 2 D - 52 )$$

The covariance matrix of the error Pₖ_{ₜ}_{+1,k}_{ₜ} can be predicted

Pₖ_{ₜ}_{+1,k}_{ₜ}=Ã⁻¹Pₖ_{ₜ}_{,k}_{ₜ}A⁻¹^{^{T}}+\tilde{Q} (2D-53)

Using these expressions, a one-step prediction from the equation of state is performed. Only a past sample is retained to predict future system behavior, through the state vector and variance-covariance matrix.

Although described in relation to transcutaneous CO₍₂₎ measurement, the invention can be implemented in other applications, so as to measure a gas emitted by a medium, whether solid or liquid. In particular, the medium may be:
a plant, for example a fruit, in order to follow the ripening process;
of slurry, so as to monitor the emission of gases such as methane;
a culture medium for biological organisms or microorganisms;
water, e.g. freshwater or seawater, e.g. to monitor gas concentration or regulate CO₂ concentration;
a floor, so as to study its breathing.

1. A method for estimating a content of a gas of interest in a medium, using a measuring device, to be arranged against the medium, the measuring device extending between a contact face, to be applied against the medium and a distal end, the measuring device comprising a side wall, extending between the contact face and the distal end, the measuring device comprising:
at the contact face, at least one inlet opening, configured to collect the gas of interest emitted by the medium, the inlet opening being made through the contact face;
a measurement chamber, including a gas sensor, the gas sensor being configured to measure a concentration of gas of interest flowing through the measurement chamber;
a collection chamber connected to the measurement chamber and delimited by an opening in the side wall, the collection chamber having at least one side opening through the side wall or in an upper wall of the collection chamber for admitting ambient air into the collection chamber;
wherein the measurement chamber is located between the contact face and the collection chamber, and the device includes drive means configured to drive air, admitted through the side opening, and the gas of interest, by convection, through the collection chamber to a discharge opening, the air convection inducing transport of the gas of interest, by diffusion, from the contact face to the collection chamber, through the measurement chamber, the method comprising:
(a) measuring the concentration of the gas of interest in the measurement chamber;
(b) by means of a processing circuit, modeling of the transport of the gas of interest between the medium and the collection chamber, the modeling taking into account the diffusion of the gas of interest from the medium to the collection chamber through the contact face, through the measurement chamber, as well as convection of the gas of interest in the collection cell resulting from the entrainment produced by the entrainment means; and
(c) from the measurement resulting from step (a), and taking into account the model resulting from step (b), estimation of the content of the gas of interest in the medium;
wherein
the device is spatially discretized according to a spatial mesh, defining mesh points between the contact face and the collection chamber; and
the model is a discretized spatio-temporal model, so as to estimate a gas content of interest at different points in the mesh, and at different times.

2. The method according to claim 1, wherein step (b) further comprises:
modeling the diffusion of the gas of interest through the measurement chamber; and
modeling the diffusion and convection of the gas of interest in the collection chamber.

3. The method according to claim 1, wherein the step (b) includes modeling the transport of the gas of interest in the medium.

4. The method according to claim 1, wherein the step (c) implements a recursive estimator.

5. The method according to claim 4, wherein the step (c) implements a linear recursive estimator.

6. The method according to claim 5, wherein the linear recursive estimator is a Kalman filter.

7. The method according to claim 1, wherein the medium is the skin of a user, the skin extending over a blood vessel, wherein the step (b) further comprises:
(i) from the gas concentration of interest in the measurement chamber resulting from step a), estimation of a transcutaneous gas concentration of interest; and
(ii) from the transcutaneous gas concentration of interest resulting from sub-step (i), estimation of a concentration or partial pressure of dissolved gas of interest in the user's blood.

8. The method according to claim 1, further comprising modeling the transport of the gas of interest emitted by the medium through the device.

9. The method according to claim 1, wherein the medium to be analyzed is a solid or liquid medium.

10. The method according to claim 1, wherein the modeling is based on a two-dimensional model, the medium, the measurement chamber and the collection chamber being discretized according to different mesh points distributed along an axis parallel to the contact face and an axis perpendicular to the contact face.
